SEQUENCE LISTING (23 sequences)

SEQ ID NO: 1 - DNA, 25 bp, Artificial Sequence
  Description of Artificial Sequence Oligonucleotide primer
accaactgggacgatatggagaaga

SEQ ID NO: 2 - DNA, 25 bp, Artificial Sequence
  Description of Artificial Sequence Oligonucleotide primer
tacgaccagaggcatacagggacaa

SEQ ID NO: 3 - DNA, 21 bp, Artificial Sequence
  Description of Artificial Sequence Oligonucleotide primer
cagccgtgcaacaatctgaag

SEQ ID NO: 4 - DNA, 21 bp, Artificial Sequence
  Description of Artificial Sequence Oligonucleotide primer
ctgcatcagtgacggtaaacc

SEQ ID NO: 5 - DNA, 20 bp, Artificial Sequence
  Description of Artificial Sequence Oligonucleotide primer
aacatcatccctgcatccac

SEQ ID NO: 6 - DNA, 20 bp, Artificial Sequence
  Description of Artificial Sequence Oligonucleotide primer
ccctgttgctgtagccgtat

SEQ ID NO: 7 - DNA, 20 bp, Artificial Sequence
  Description of Artificial Sequence Oligonucleotide primer
gccgatccacacggagtact

SEQ ID NO: 8 - DNA, 18 bp, Artificial Sequence
  Description of Artificial Sequence Oligonucleotide primer
ctggcacccagcacaatg

SEQ ID NO: 9 - DNA, 20 bp, Artificial Sequence
  Description of Artificial Sequence Oligonucleotide primer
gtgtcactggcgacacgtag

SEQ ID NO: 10 - DNA, 20 bp, Artificial Sequence
  Description of Artificial Sequence Oligonucleotide primer
tcccatcccacagagagaag

SEQ ID NO: 11 - DNA, 20 bp, Artificial Sequence
  Description of Artificial Sequence Oligonucleotide primer
ccattagaaagcagttccgc

SEQ ID NO: 12 - DNA, 20 bp, Artificial Sequence
  Description of Artificial Sequence Oligonucleotide primer
tgggtaggagatggagatgc

SEQ ID NO: 13 - DNA, 20 bp, Artificial Sequence
  Description of Artificial Sequence Oligonucleotide primer
aacatcatccctgcctctac

SEQ ID NO: 14 - DNA, 20 bp, Artificial Sequence
  Description of Artificial Sequence Oligonucleotide primer
ccctgttgctgtagccaaat

SEQ ID NO: 15 - DNA, 20 bp, Artificial Sequence
  Description of Artificial Sequence Oligonucleotide primer
tctaacggccaaagtggttt

SEQ ID NO: 16 - DNA, 20 bp, Artificial Sequence
  Description of Artificial Sequence Oligonucleotide primer
gccacctctctgtgtccttc

SEQ ID NO: 17 - DNA, 30 bp, Artificial Sequence
  Description of Artificial Sequence Oligonucleotide primer
cgcggatccggcccacagccatctaacggc

SEQ ID NO: 18 - DNA, 32 bp, Artificial Sequence
  Description of Artificial Sequence Oligonucleotide primer
ccggaattcgcaatgcggctgacggagagtga

SEQ ID NO: 19 - DNA, 27 bp, Artificial Sequence
  Description of Artificial Sequence Oligonucleotide primer
gcgggtaccctaatgcagccgctgacc

SEQ ID NO: 20 - DNA, 27 bp, Artificial Sequence
  Description of Artificial Sequence Oligonucleotide primer
gcggctagcctttgggcctcgctttgt

SEQ ID NO: 21 - DNA, 29 bp, Artificial Sequence
  Description of Artificial Sequence Oligonucleotide primer
gcgggtaccctgcttgtcagacacgatgc

SEQ ID NO: 22 - DNA, 29 bp, Artificial Sequence
  Description of Artificial Sequence Oligonucleotide primer
gcggctagccctcagtttcctcgcctgta

SEQ ID NO: 23 - DNA, 29 bp, Artificial Sequence
  Description of Artificial Sequence Oligonucleotide primer
cctgccctggggatttttccctgtccctg

(19) United States
(12) Patent Application Publication
(10) Pub. No.: US 2006/0154860 A1
(43) Pub. Date: Jul. 13, 2006

(54) Manipulating stem-progenitor cell trafficking to injured tissue and/or tumors by altering HIF-1 and/or SDF-1 activity

(76) Inventors: Daniel J. Ceradini, New York, NY (US); Geoffrey C. Gurtner, New York, NY (US)

Correspondence Address: Michael L. Goldman, Esq.;Nixon Peabody LLP, Clinton Square, P.O. Box 31051, Rochester, NY, 14603-1051, US

(21) Appl. No.: 11/187,585

(22) Filed: Jul. 22, 2005

Related U.S. Application Data
(60) Provisional application No. 60/590,077, filed on Jul. 22, 2004.

(51) Int. Cl. A61K 48/00; A61K 38/18

(52) U.S. Cl. 514/12; 514/44

(57) ABSTRACT

The present invention relates to a method for modulating recruitment of stem cells or progenitor cells to a selected tissue site. Methods for treating damaged tissue and for treating cancerous tumor tissue are also disclosed. These methods involve regulating HIF-1 and/or SDF-1 activity in the tissue.

This application claims the benefit of U.S. Provisional Patent Application Ser. No. 60/590,077, filed Jul. 22, 2004.

This invention was developed with government funding under grant EB002265 from the National Institutes of Health/National Institute for Biomedical Imaging and Bioengineering (G.C.G.). The United States Government may have certain rights in this invention.

### FIELD OF THE INVENTION

This invention is directed generally to methods involving altering activity of HIF-1 and/or SDF-1 to modulate recruitment of stem and progenitor cells.

### BACKGROUND OF THE INVENTION

After injury, the body must be able to specifically identify areas that need repair in order to heal itself. Otherwise the body's reparative machinery would always be turned “on” without knowing when and where injury has occurred. Tissue repair and regeneration following injury is believed to be mediated by stem and progenitor cells that are either recruited from circulating blood, or already resident in tissues (Kollet et al., “HGF, SDF-1, and MMP-9 are Involved in Stress-induced Human CD34⁺ Stem Cell Recruitment to the Liver,” J. Clin. Invest. 112:160-169 (2003)).

The trafficking of circulating stem and progenitor cells to areas of tissue damage is poorly understood. Stromal cell-derived factor-1 (SDF-1, CXCL12) is a multifunctional chemokine which mediates the homing of stem cells to bone marrow via binding of CXCR4 on circulating cells (Peled et al., “Dependence of Human Stem Cell Engraftment and Repopulation of NOD/SCID Mice on CXCR4,” Science 283:845-848 (1999); Peled et al., “The Chemokine SDF-1 Stimulates Integrin-mediated Arrest of CD34⁺ Cells on Vascular Endothelium Under Shear Flow,” J. Clin. Invest. 104:1199-1211 (1999)). SDF-1 and CXCR4 are also expressed in complementary patterns during embryonic organogenesis and guide primordial stem cells to sites of rapid vascular expansion (McGrath et al., “Embryonic Expression and Function of the Chemokine SDF-1 and its Receptor, CXCR4,” Dev. Biol. 213:442-456 (1999)). The importance of SDF-1 in stem and progenitor cell recruitment has been established with recent observations that selective expression in injured tissue correlates with adult stem cell recruitment and tissue regeneration (Askari et al., “Effect of Stromal-cell-derived Factor 1 on Stem-cell Homing and Tissue Regeneration in Ischaemic Cardiomyopathy,” Lancet 362:697-703 (2003); Yamaguchi et al., “Stromal Cell-derived Factor-1 Effects on Ex Vivo Expanded Endothelial Progenitor Cell Recruitment for Ischemic Neovascularization,” Circulation 107:1322-1328 (2003)).

However, the regulation of SDF-1 expression and its physiologic role in peripheral tissue repair remains unknown (Kollet et al., “HGF, SDF-1, and MMP-9 are Involved in Stress-induced Human CD34⁺ Stem Cell Recruitment to the Liver,” J. Clin. Invest. 112:160-169 (2003)). The physiologic mechanism underlying the localized expression of SDF-1 in injured tissue is completely unknown. Many factors produced during tissue injury could potentially regulate SDF-1 expression including inflammatory mediators (interleukin-1, tumor-necrosis factor-α), changes in the extracellular matrix and altered mechanical forces.

What is needed is a method of modulating recruitment of stem and progenitor cells to selected tissue sites. The present invention is directed to overcoming these and other deficiencies in the art.

### SUMMARY OF THE INVENTION

One aspect of the present invention relates to a method of treating damaged tissue. This method involves increasing HIF-1 and/or SDF-1 activity in damaged tissue to a level above that caused by damage alone to the tissue, under conditions effective to treat the damaged tissue.

Another aspect of the present invention relates to a method of modulating recruitment of stem cells and/or progenitor cells to a selected tissue site. This method involves controlling HIF-1 and/or SDF-1 activity at the selected tissue site to a level different than activity otherwise present at the selected tissue site, under conditions effective to modulate recruitment of stem cells and/or progenitor cells to the selected tissue site.

Another aspect of the present invention relates to a method of treating cancer in a subject. This method involves limiting HIF-1 and/or SDF-1 activity in cancerous tumor tissue in the subject under conditions effective to treat the subject's cancer.

While the use of stem/progenitor cells for therapeutic purposes has rapidly progressed (e.g., Schachinger et al, “Transplantation of Progenitor Cells and Regeneration Enhancement in Acute Myocardial Infarction Final One-year Results of the TOPCARE-AMI Trial,” J. Am. Coll. Cardiol. 44:1690-1699 (2004); Strauer et al., “Repair of Infarcted Mycocardium by Autologous Intracoronary Mononuclear Bone Marrow Cell Transplantation in Humans,” Circulation 106:1913-1918 (2002); Tateishi-Yuyuma et al., “Therapeutic Angiogenesis for Patients with Limb Ischaemia by Autologous Transplantation of Bone-marrow Cells: A Pilot Study and a Randomised Controlled Trial,” Lancet 360:427435 (2002), which are hereby incorporated by reference in their entirety), the mechanism by which progenitor cells are able to selectively home to injured or ischemic tissues remained unclear. Advantageously, the present invention identifies mechanisms regulating selective mobilization and recruitment of stem/progenitor cells, thus enabling clinicians to augment therapeutic targeting of reparative stem/progenitor cells in the clinical setting.

describes a novel way to modulate stem and/or progenitor cell recruitment, and to treat damaged tissue and cancerous tumors.

### BRIEF DESCRIPTION OF THE DRAWINGS

FIGS. 1A-B are a schematic diagram (FIG. 1A) and photograph (FIG. 1B) of a nude mouse demonstrating increasingly ischemic tissue areas (labeled A-C), determined by direct oxygen tension measurements at points p1-p4 and laser Doppler 12 hours postoperatively.

FIG. 2 is a graph illustrating oxygen tension at p1-p4 and non-ischemic tissue (N) after 12 hours, 7 days, and 14 days postoperatively.

FIGS. 3A-D are hematoxylin and eosin (“H&E”) stained images of mouse epithelial tissue 14 days postoperatively (100×). FIG. 3A is an H&E of non-ischemic tissue. FIGS. 3B, 3C, and 3D are H&E of tissue taken from tissue areas A, B, and C (see FIG. 1A), respectively. Mild perivascular infiltration of monocyte-like cells in more ischemic regions (inset 200×, FIGS. 3C and 3D) and a slight increase in dermal cellularity (asterisk, FIG. 3D) accompanies increased capillary density (black arrowheads).

FIGS. 4A-B are graphs illustrating SDF-1 transcription and expression. FIG. 4A illustrates quantitative real time RT-PCR from total RNA harvested from non-ischemic tissue (N) and ischemic tissue areas A-C (see FIG. 1A) 6 hours postoperatively. Mean fold induction (F.I.) over non-ischemic levels are indicated at right. FIG. 4B illustrates SDF-1 protein expression at 12 hours postoperatively.

FIGS. 5A-C are stained images (200×) showing SDF-1 (indicated by arrows) expression in vivo in non-ischemic tissue (FIG. 5A), Area A (FIG. 5B), and Area C (FIG. 5C) at 12 hours post-surgery. Vessels were stained green by FITC-lectin perfusion.

FIGS. 6A-B are stained images (400×) of axial sectioned blood vessels from non-ischemic tissue (FIG. 6A) and Area C (FIG. 6B). Double arrows indicate SDF-1 immunostaining.

FIG. 7 is a stained image (400×) of ischemic tissue (Area C) showing the co-localization (asterisk) of the endothelial marker CD31 and SDF-1.

FIGS. 8A-F are stained images showing SDF-1 mRNA in situ hybridization in non-ischemic (FIGS. 8A-B) and ischemic (FIGS. 8C-F) (Area C) tissue. FIGS. 8A and 8C are H&E images (200×) of serial sections. White arrowheads indicate vessels. FIGS. 8B and 8D are in situ mRNA hybridization images (200×) of the serial sections shown in FIGS. 8A and 8C, respectively. Black arrowheads (indicating purple/dark blue staining) indicate endothelial cell localization of SDF-1 mRNA. FIGS. 8E-F are stained images (400×) of Area C showing SDF-1 mRNA expression by ischemic blood vessel endothelium (double black arrowheads). Little or no expression is seen in smooth muscle (double open arrowheads), pericytes (single open arrowhead), and surrounding stromal cells. FIG. 8E shows H&E staining with no immunostaining.

FIG. 9 is a graph of an ELISA of culture supernatants (HUVEC) after 6, 12, and 18 hours of hypoxia (H6, H12, H18) compared to normoxia (N) (*=P<0.005).

FIG. 10 is a graph of surface bound SDF-1 during hypoxic culture media (shaded) compared to normoxia (open).

FIG. 11 is a graph of SDF-1 mRNA transcripts under normoxic (N) and hypoxic (H3, H6, H9) conditions (*=P<0.005).

FIG. 12 is a schematic diagram of the human SDF1 locus showing two potential HIF-1 binding site sequences (HBS1 and HBS2) and the transcriptional start site (0).

FIG. 13 is a schematic diagram (left) and graph (right) illustrating the luciferase activity of pGL3b.SDF1.full, serial 5′ deletions (pGL3b.SDF1.-553 and pGL3b.SDF1.-441), and mutation of HBS 1 (pGL3b.SDF1.MUT.HBS1).

FIG. 14 is a schematic diagram (left) and graph (right) illustrating the fold induction of luciferase activity of the potential HIF-1 binding sites (HBS1 and HBS2) inserted 5′ of the minimal SV40 promoter.

FIG. 15 is a graph illustrating the fold induction of relative luciferase activity under normal (open bar) and hypoxic (shaded bar) conditions of pGL3b.SDF1.full (top two bars) and pGL3b.SDF1.full co-transfected with HIF-1.ΔODD or HIF-1.DN.

FIGS. 16A-B are graphs illustrating the effect of HIF-1α gene silencing on HIF-1α (FIG. 16A) and SDF-1 (FIG. 16B) mRNA expression (*=P<0.005, **=P<0.01).

FIGS. 17A-B are a graph (FIG. 17A) and a gel (FIG. 17B) showing chromatin immunoprecipitation of SDF1 specific genomic sequences from endothelial cells cultivated in normoxic (N) or hypoxic (H) conditions using HIF-1α monoclonal antibodies (M: marker, +: plasmid control).

FIGS. 18A-B are stained images (400×) of two vessels showing in vivo co-localization (white arrow heads) of HIF-1α and SDF-1 in post-capillary venules of ischemic tissue.

FIGS. 19A-D are graphs illustrating the high expression levels of CXCR4 (FIG. 19A), KDR/FLK-1 (FIG. 19B), and CD31 (FIG. 19C), and CD31/CXCR4 (FIG. 19D) in EPCs, with >94% co-expressing CD31/CXCR4. Functional interactions between endothelial cells and progenitor cells mediated by HIF-1 induced SDF-1 expression.

FIG. 20 is a graph illustrating the percent adherence of CXCR4⁺ EPCs to normoxic and hypoxic HUVEC monolayers in the presence and absence of antibodies specific for SDF-1 (SDF-1 ab), EPC CXCR4 (EPC CXCR4 ab), and HUVEC CXCR4 (HUVEC CXCR4 ab). Hypoxia-conditioned HUVEC monolayers adhere significantly more EPCs, an effect specific to SDF-1/CXCR4 interactions (*=P<0.001).

FIG. 21 is a graph illustrating the adherence of CXCR4⁺ EPCs in normoxic (open bars) and hypoxic (shaded bars) conditions to normal HUVEC monolayers (CTRL) and HUVEC monolayers in which HIF-1α expression was silenced (HIF-1α siRNA). Silencing of HIF-1α in HUVECs abolished hypoxia-specific adhesion (*=P<0.005).

FIG. 22 is a graph illustrating the percent adherence of CXCR4⁺ EPCs to normoxic HUVEC monolayers and normoxic HUVEC monolayers that were pre-coated for 20 minutes with recombinant SDF-1 (rSDF-1). Adherence was measured in the presence and absence of antibodies specific for EPC CXCR4 (EPC CXCR4 ab) and HUVEC CXCR4 (HUVEC CXCR4 ab). Normoxic HUVEC monolayers adhered more EPCs after being coated with SDF-1 (*=P<0.001).

FIG. 23 is a graph illustrating the percent adherence to normoxic HUVEC monolayers of normal CXCR4⁺ EPCs (N) and CXCR4⁺ EPCs preincubated with soluble SDF-1 for 20 minutes prior to assay (+EPC rSDF-1). Preincubation of EPCs with SDF-1 prior to assay reduced baseline adhesion (*=P<0.005).

FIG. 24 is a graph illustrating the migration of CXCR4⁺ EPCs in, from left, media alone (M), media with recombinant SDF-1 (M+S), normoxia-conditioned media (NM), hypoxia-conditioned media (HM), and hypoxia-conditioned media preincubated with neutralizing CXCR4 antibodies (HM+ab). EPC migration increased in response to media with recombinant SDF-1 compared to media alone (M versus M+S). Hypoxia-conditioned media from HUVECs stimulated an increase in migrating EPCs as compared to normoxic media (NM versus HM), which could be blocked by preincubating EPCs with neutralizing CXCR4 antibody(HM+ab). *=P<0.001; **=P<0.001; ***=P<0.001.

FIG. 25 is stained images (200×) of ischemic tissue 14 days postoperatively showing EPC engraftment in Areas A-C. DiI-labeled EPCs engrafted ischemic tissue in proportion to reduced oxygen tension (vessels stained with FITC-lectin).

FIG. 26 is a graph quantifying and comparing EPC engraftment in Areas A-C shown in FIG. 25 and in non-ischemic tissue (NI).

FIGS. 27A-B are stained images (400×) of non-ischemic (FIG. 27A) and ischemic (FIG. 27B) tissue. EPCs (arrows) were very rarely identified in non-ischemic tissue lining blood vessels, and were frequently found in ischemic tissue lining functional microvascular channels.

FIGS. 28A-C are stained images (200×) of ischemic tissue showing homing and engraftment of CXCR4⁺ EPCs in control tissue (FIG. 28A), in tissue preincubated with neutralizing CXCR4 antibody prior to administration (FIG. 28B), and with administration with free neutralizing SDF-1 antibodies (FIG. 28C).

FIG. 29 is a graph comparing the EPC homing and engraftment shown in FIGS. 28A-C. Blockade of EPC CXCR4 or co-administration of free neutralizing SDF-1 antibodies significantly reduced the number of EPCs that engrafted ischemic tissue (*=P<0.0001).

FIGS. 30A-C are a schematic diagram (FIG. 30A) and graphs (FIGS. 30B-C). FIG. 30A shows Doppler blood flow in ischemic tissue after 14 days in mice treated with saline (S) EPCs (E), EPCs preincubated with CXCR4 antibody (E/C), and EPCs co-administered with SDF-1 antibody (E/S). FIGS. 30B-C show the capillary density (FIG. 30B) (*=P<0.001) and relative blood flow (FIG. 30C) (*=P<0.01) in Area C of each group illustrated in FIG. 30A (n=4).

FIG. 31 is a graph illustrating the oxygen tensions of the bone marrow, non-ischemic tissues, and ischemic tissue in the nude mouse model described in Example 21 (p1-4).

FIGS. 32A-B are DAPI stained images (200×) of a section of bone marrow. FIG. 32A delineates bone marrow compartment (BM), cortical bone (C), and non-ischemic periosteal tissue (P). FIG. 32B shows the same section with co-localization of pimonidazole and SDF-1 immunostaining (indicated by arrows).

FIGS. 33A-B are DAPI stained images (200×) of a longitudinal section of bone marrow. FIG. 33B demonstrates EPC localization (indicated by white arrowheads) to SDF-1 rich regions following intravascular administration.

### DETAILED DESCRIPTION OF THE INVENTION

The present invention relates to a method of treating damaged tissue. This method involves increasing HIF-1 and/or SDF-1 activity in damaged tissue to a level above that caused by damage alone to the tissue, under conditions effective to treat the damaged tissue.

HIF-1 is the heterodimeric protein product of the HIF1 gene (Wang et al., “Hypoxia-inducible Factor 1 is a Basic-helix-loop-helix-PAS Heterodimer Regulated by Cellular O₂ Tension,” Proc. Nat'l Acad. Sci. U.S.A. 92(12): 5510-5514 (1995) (setting forth the amino acid sequence of HIF-1α); see also GenBank Accession No. U22431 (H. sapiens HIF-1α mRNA); GenBank Accession No. AJ277829 (X. laevis HIF-1α mRNA); GenBank Accession No. NM_{—}075607 (C. elegans HIF-1 complete mRNA); GenBank Accession No. AJ715791 (S. judaei HIF-1α mRNA); GenBank Accession No. AY621118 (B. grunniens HIF-1α complete mRNA); GenBank Accession No. AY450269 (C. idella HIF-1 complete mRNA); GenBank Accession No. AF304864 (O. mykiss HIF-1α complete mRNA); GenBank Accession No. Q16665 (H. sapiens HIF-1α amino acid sequence); GenBank Accession No. AAL27308 (H. sapiens HIF-1 amino acid sequence); which are hereby incorporated by reference in their entirety).

SDF-1 is a chemokine, the protein product of the SDF1 gene (Bleul et al, “A Highly Efficacious Lymphocyte Chemoattractant, Stromal Cell-derived Factor 1 (SDF-1),” J. Exp. Med. 184:1101-1109; see also GenBank Accession No. AJ278857 (X. laevis SDF-1 mRNA); GenBank Accession No. E09670 (mouse SDF-1α cDNA); GenBank Accession No. E09669 (human SDF-1β cDNA); GenBank Accession No. E09668 (human SDF-1α cDNA); GenBank Accession No. AF209976 (R. norvegicus SDF-1 complete mRNA); GenBank Accession No. U16752 (human SDF-1β complete mRNA); GenBank Accession No. L12030 (M. musculus SDF-1β complete mRNA); GenBank Accession No. L12029 (M. musculus SDF-1α complete mRNA); GenBank Accession No. AAH61945 (X. laevis SDF-1 protein); GenBank Accession No. AAA97434 (human SDF-1β protein); which are hereby incorporated by reference in their entirety).

HIF-1 and SDF-1 increase the adhesion, migration, and homing of progenitor cells, which repair and regenerate tissue. SDF-1 gene expression is regulated by the transcription factor hypoxia-inducible factor-1 (HIF-1) in endothelial cells. HIF-1α alone can induce SDF-1 expression. Blockade of SDF-1 in ischemic tissue or CXCR4 on circulating cells prevents progenitor cell recruitment to sites of injury. Recruitment of CXCR4⁺ progenitor cells to regenerating tissues is mediated by hypoxic gradients via HIF-1 induction of SDF-1 expression.

The methods of the present invention may be carried out in vitro or in vivo.

The methods of the present invention involve regulating HIF-1 and/or SDF-1 activity. Regulation includes up-regulation (increasing activity) and down-regulation (decreasing or limiting activity), and may be carried out by, e.g., regulating expression of HIF-1 and/or SDF-1, administering HIF-1 and/or SDF-1, and/or administering agents that modulate HIF-1 and/or SDF-1 activity.

HIF-1 and SDF-1 expression may be up-regulated by, for example, growth factors, e.g., insulin-like growth factor 1 (IGF-1), vascular endothelial growth factor (VEGF), and platelet-derived growth factor (PDGF); and hypoxia.

Expression of SDF-1 may also be regulated by, for example, regulating HIF-1 activity. Increasing HIF-1 activity increases SDF-1 expression; decreasing HIF-1 activity decreases SDF-1 expression (see e.g., Examples 17 and 18 infra).

HIF-1 and/or SDF-1 activity in a tissue may also be increased by, for example, administering HIF-1 and/or SDF-1 to the tissue. HIF-1 and/or SDF-1 may be administered by introducing to the tissue a nucleic acid molecule encoding the desired protein. Alternatively or additionally, HIF-1 and/or SDF-1 may be administered by introducing to the tissue the desired protein.

In all aspects of the present invention involving introducing a nucleic acid to tissue, the nucleic acid molecule of choice can be introduced into an expression system or vector of choice using conventional recombinant technology. Generally, this involves inserting the nucleic acid molecule into an expression system to which the molecule is heterologous (i.e., not normally present). One or more desired nucleic acid molecules may be inserted into the vector. When multiple nucleic acid molecules are inserted, the multiple nucleic acid molecules may encode the same or different proteins. The heterologous nucleic acid molecule is inserted into the expression system or vector in proper sense (5′→3′) orientation and correct reading frame when expression of the encoded HIF-1 and/or SDF-1 protein in the tissue is desired. Alternatively, the nucleic acid may be inserted in the “antisense” orientation, i.e, in a 3′→5′ prime direction, such that antisense RNA is produced. In each of these aspects, the vector contains the necessary elements for the transcription and translation of the inserted HIF-1 and/or SDF-1 protein-coding sequences. The orientation of the nucleic acid molecule will be dependent on whether the regulation is intended to be a downregulation or an upregulation. Where upregulation is intended, a suitable nucleic acid molecule is inserted in the sense orientation to allow expression of an HDF-1 and/or SDF-1 protein capable of effecting the methods of the present invention (e.g., capable of increasing stem/progenitor cell recruitment). When the intended regulation is a downregulation, a suitable nucleic acid molecule may be inserted in the antisense orientation.

Antisense nucleic acids are DNA molecules, RNA molecules, oligoribonucleotides, or oligodeoxyribonucleotides that are complementary to at least a portion of a specific mRNA molecule (Weintraub, Scientific American 262:40 (1990), which is hereby incorporated by reference in its entirety). In the cell, the antisense nucleic acids are transcribed and hybridize to a target nucleic acid. The specific hybridization of an antisense nucleic acid molecule with its target nucleic acid interferes with the normal function of the target nucleic acid. The functions of DNA to be interfered with include replication and transcription. The functions of RNA to be interfered with include all vital functions, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is the regulation of the protein expression.

In aspects of the present invention in which down-regulation of HIF-1 and/or SDF-1 activity is desired, the method of interfering with endogenous protein expression may involve an RNA-based form of gene-silencing known as RNA-interference (RNAi) (also known more recently as siRNA for short, interfering RNAs). RNAi is a form of post-transcriptional gene silencing (PTGS). PTGS is the silencing of an endogenous gene caused by the introduction of a homologous double-stranded RNA (dsRNA), transgene, or virus. In PTGS, the transcript of the silenced gene is synthesized, but does not accumulate because it is degraded. RNAi is a specific from of PTGS, in which the gene silencing is induced by the direct introduction of dsRNA. Numerous reports have been published on critical advances in the understanding of the biochemistry and genetics of both gene silencing and RNAi (Matzke et al., “RNA-Based Silencing Strategies in Plants,” Curr Opin Genet Dev 11 (2):221-227 (2001), Hammond et al., “Post-Transcriptional Gene Silencing by Double-Stranded RNA,” Nature Rev Gen 2:110-119 (Abstract) (2001); Hamilton et al., “A Species of Small Antisense RNA in Post-transcriptional Gene Silencing in Plants,” Science 286:950-952 (Abstract) (1999); Hammond et al., “An RNA-Directed Nuclease Mediates Post-Transcriptional Gene Silencing in Drosophila Cells,” Nature 404:293-298 (2000); Hutvagner et al., “RNAi: Nature Abhors a Double-Strand,” Curr Opin Genetics & Development 12:225-232 (2002), which are hereby incorporated by reference in their entirety). In iRNA, the introduction of double stranded RNA (dsRNA) into animal or plant cells leads to the destruction of the endogenous, homologous mRNA, phenocopying a null mutant for that specific gene. In siRNA, the dsRNA is processed to short interfering molecules of 21-, 22- or 23-nucleotide RNAs (siRNA), which are also called “guide RAs,” (Hammond et al., “Post-Transcriptional Gene Silencing by Double-Stranded RNA,” Nature Rev Gen 2:110-119 (Abstract) (2001); Sharp, P. A., “RNA Interference-2001,” Genes Dev 15:485-490 (2001); Hutvagner et al., “RNAi: Nature Abhors a Double-Strand,” Curr Opin Genetics & Development 12:225-232 (2002), which are hereby incorporated by reference in their entirety) in vivo by the Dicer enzyme, a member of the RNAse III-family of dsRNA-specific ribonucleases (Hutvagner et al., “RNAi: Nature Abhors a Double-Strand,” Curr Opin Genetics & Development 12:225-232 (2002); Bernstein et al., “Role for a Bidentate Ribonuclease in the Initiation Step of RNA Interference,” Nature 409:363-366 (2001); Tuschl, T., “RNA Interference and Small Interfering RNAs,” Chembiochem 2: 239-245 (2001); Zamore et al., “RNAi: Double Stranded RNA Directs the ATP-Dependent Cleavage of mRNA at 21 to 23 Nucleotide Intervals,” Cell 101:25-3 (2000); U.S. Pat. No. 6,737,512 to Wu et al., which are hereby incorporated by reference in their entirety). Successive cleavage events degrade the RNA to 19-21 bp duplexes, each with 2-nucleotide 3′ overhangs (Hutvagner et al., “RNAi: Nature Abhors a Double-Strand,” Curr Opin Genetics & Development 12:225-232 (2002); Bernstein et al., “Role for a Bidentate Ribonuclease in the Initiation Step of RNA Interference,” Nature 409:363-366 (2001), which are hereby incorporated by reference in their entirety). The siRNAs are incorporated into an effector known as the RNA-induced silencing complex (RISC), which targets the homologous endogenous transcript by base pairing interactions and cleaves the mRNA approximately 12 nucleotides form the 3′ terminus of the siRNA (Hammond et al., “Post-Transcriptional Gene Silencing by Double-Stranded RNA,” Nature Rev Gen 2:110-119 (Abstract) (2001); Sharp, P. A., “RNA Interference-2001,” Genes Dev 15:485-490 (2001); Hutvagner et al., “RNAi: Nature Abhors a Double-Strand,” Curr Opin Genetics & Development 12:225-232 (2002); Nykanen et al., “ATP Requirements and Small Interfering RNA Structure in the RNA Interference Pathway,” Cell 107:309-321 (2001), which are hereby incorporated by reference in their entirety).

There are several methods for preparing siRNA, including chemical synthesis, in vitro transcription, siRNA expression vectors, and PCR expression cassettes. In one aspect of the present invention, dsRNA for HIF-1 and/or SDF-1 nucleic acid molecules can be generated by transcription in vivo. This involves modifying the HIF-1 and/or SDF-1 nucleic acid molecule for the production of dsRNA, inserting the modified nucleic acid molecule into a suitable expression vector having the appropriate 5′ and 3′ regulatory nucleotide sequences operably linked for transcription and translation and introducing the expression vector having the modified nucleic acid molecule into a suitable host or subject. Using siRNA for gene silencing is a rapidly evolving tool in molecular biology, and guidelines are available in the literature for designing highly effective siRNA targets and making antisense nucleic acid constructs for inhibiting endogenous protein (U.S. Pat. No. 6,737,512 to Wu et al.; Brown et al., “RNA Interference in Mammalian Cell Culture: Design, Execution, and Analysis of the siRNA Effect,” Ambion TechNotes 9(l):3-5(2002); Sui et al., “A DNA Vector-Based RNAi Technology to Suppress Gene Expression in Mammalian Cells,” Proc Natl Acad Sci USA 99(8):5515-5520 (2002); Yu et al., “RNA Interference by Expression of Short-Interfering RNAs and Hairpin RNAs in Mammalian Cells,” Proc Natl Acad Sci USA 99(9):6047-6052 (2002); Paul et al., “Effective Expression of Small Interfering RNA in Human Cells,” Nature Biotechnology 20:505-508 (2002); Brummelkamp et al., “A System for Stable Expression of Short Interfering RNAs in Mammalian Cells,” Science 296:550-553 (2002), which are hereby incorporated by reference in their entirety). There are also commercially available sources for custom-made siRNAs. Suitable methods for HIF silencing include, for example, those described in Berra, “HIF Prolyl-hydroxylase 2 is the Key Oxygen Sensor Setting Low Steady-state Levels of HIF-1α in Normoxia,” EMBO J. 22(16):4082-4090 (2003), which is hereby incorporated by reference in its entirety.

The preparation of the nucleic acid constructs that include a nucleic acid molecule suitable to regulate HIF-1 and/or SDF-1 activity may be carried out using methods well known in the art. U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture. Other vectors are also suitable.

Suitable vectors include, but are not limited to, vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKCO101, SV 40, pBluescript II SK +/− or KS +/− (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Human gene therapy is an approach to treating human disease that is based on the modification of gene expression in cells of the patient. Eukaryotic viruses have been employed as vehicles for somatic gene therapy. Among the viral vectors that have been cited frequently in gene therapy research are adenoviruses (U.S. Pat. No. 6,203,975 to Wilson). Several viral systems including murine retrovirus, adenovirus, parvovirus (adeno-associated virus), vaccinia virus, and herpes virus have been developed as therapeutic gene transfer vectors (for review see, Nienhuis et al., Hematology, Vol. 16: Viruses and Bone Marrow, N. S. Young (ed.), pp. 353-414 (1993), which is hereby incorporated by reference in its entirety). Viral vectors provide a more efficient means of transferring genes into cells as compared to other techniques such as calcium phosphate or DEAE-dextran-mediated transfection, electroporation, or microinjection. It is believed that the efficiency of viral transfer is due to the fact that the transfer of DNA is a receptor-mediated process (i.e., the virus binds to a specific receptor protein on the surface of the cell to be infected).

Once a suitable expression vector is selected, the desired nucleic acid sequences are cloned into the vector using standard cloning procedures in the art, as described by Sambrook et al., Molecular Cloning: A Laboratory Manual, Cold Springs Laboratory, Cold Springs Harbor, N.Y. (1989), or U.S. Pat. No. 4,237,224 to Cohen and Boyer, which are hereby incorporated by reference in their entirety. The vector is then introduced to a suitable host.

A variety of host-vector systems may be utilized to express the recombinant HIF-1/SDF-1 protein inserted into a vector as described above. Primarily, the vector system must be compatible with the host used. Host-vector systems include, without limitation, the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used to carry out this and other aspects of the present invention.

Different genetic signals and processing events control many levels of gene expression (e.g., DNA transcription and messenger RNA (“mRNA”) translation). Transcription of DNA is dependent upon the presence of a promoter, which is a DNA sequence that directs the binding of RNA polymerase, and thereby promotes mRNA synthesis. The DNA sequences of eukaryotic promoters differ from those of prokaryotic promoters. Furthermore, eukaryotic promoters and accompanying genetic signals may not be recognized in, or may not function in, a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Common promoters suitable for directing expression in mammalian cells include, without limitation, SV40, MMTV, metallothionein-1, adenovirus Ela, CMV, immediate early, immunoglobulin heavy chain promoter and enhancer, and RSV-LTR.

When multiple nucleic acid molecules are inserted, the multiple nucleic acid molecules may all be placed under a single 5′ regulatory region and a single 3′ regulatory region, where the regulatory regions are of sufficient strength to transcribe and/or express the nucleic acid molecules as desired.

Specific initiation signals are also required for efficient gene transcription and translation in prokaryotic cells. These transcription and translation initiation signals may vary in “strength” as measured by the quantity of gene specific messenger RNA and protein synthesized, respectively. The nucleic acid expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (ATG) to provide a ribosome binding site. Thus, any SD-ATG combination that can be utilized by host ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used. Depending on the vector system and host utilized, any number of suitable transcription and/or translation elements, including constitutive, inducible, and repressible promoters, as well as minimal 5′ promoter elements, enhancers or leader sequences may be used.

Typically, when a recombinant host is produced, an antibiotic or other compound useful for selective growth of the transgenic cells only is added as a supplement to the media. The compound to be used will be dictated by the selectable marker element present in the plasmid with which the host was transformed. Suitable genes are those which confer resistance to gentamycin, G418, hygromycin, streptomycin, spectinomycin, tetracycline, chloramphenicol, and the like. Similarly, “reporter genes,” which encode enzymes providing for production of an identifiable compound, or other markers which indicate relevant information regarding the outcome of gene delivery, are suitable. For example, various luminescent or phosphorescent reporter genes are also appropriate, such that the presence of the heterologous gene may be ascertained visually.

An example of a marker suitable for the present invention is the green fluorescent protein (GFP) gene. The isolated nucleic acid molecule encoding a green fluorescent protein can be deoxyribonucleic acid (DNA) or ribonucleic acid (RNA, including messenger RNA or mRNA), genomic or recombinant, biologically isolated or synthetic. The DNA molecule can be a cDNA molecule, which is a DNA copy of a messenger RNA (mRNA) encoding the GFP. In one embodiment, the GFP can be from Aequorea victoria (Prasher et al., “Primary Structure of the Aequorea Victoria Green-Fluorescent Protein,” Gene 11 1(2):229-233 (1992); U.S. Pat. No. 5,491,084 to Chalfie et al., which are hereby incorporated by reference in their entirety). A plasmid encoding the GFP of Aequorea victoria is available from the ATCC as Accession No. 75547. Mutated forms of GFP that emit more strongly than the native protein, as well as forms of GFP amenable to stable translation in higher vertebrates, are commercially available from Clontech Laboratories, Inc. (Palo Alto, Calif.) and can be used for the same purpose. The plasmid designated pTα1-GFPh (ATCC Accession No. 98299, which is hereby incorporated by reference in its entirety) includes a humanized form of GFP. Indeed, any nucleic acid molecule encoding a fluorescent form of GFP can be used in accordance with the subject invention. Standard techniques are then used to place the nucleic acid molecule encoding GFP under the control of the chosen cell specific promoter.

The selection marker employed will depend on the target species and/or host or packaging cell lines compatible with a chosen vector.

A nucleic acid molecule encoding a suitable protein, a promoter molecule of choice, including, without limitation, enhancers, and leader sequences; a suitable 3′ regulatory region to allow transcription in the host, and any additional desired components, such as reporter or marker genes, are cloned into the vector of choice using standard cloning procedures in the art, such as described in Sambrook et al., Molecular Cloning: A Laboratory Manual, Cold Spring Laboratory, Cold Spring Harbor, N.Y. (1989); Ausubel et al., “Short Protocols in Molecular Biology,” New York:Wiley (1999), and U.S. Pat. No. 4,237,224 to Cohen and Boyer, which are hereby incorporated by reference in their entirety.

Once the isolated nucleic acid molecule encoding a suitable protein has been cloned into an expression vector, it is ready to be incorporated into a host. Recombinant molecules can be introduced into cells, without limitation, via transformation (if the host is a prokaryote), transfection (if the host is a eukaryote), transduction (if the host is a virus), conjugation, mobilization, or electroporation, lipofection, protoplast fusion, mobilization, particle bombardment, or electroporation, using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, virus, yeast, and mammalian cells, including, without limitation, stem cells and dendritic cells.

Transient expression in protoplasts allows quantitative studies of gene expression since the population of cells is very high (on the order of 10⁶). To deliver DNA inside protoplasts, several methodologies have been proposed, but the most common are electroporation (Neumann et al., “Gene Transfer into Mouse Lyoma Cells by Electroporation in High Electric Fields,” EMBO J 1:841-45 (1982); Wong et al., “Electric Field Mediated Gene Transfer,” Biochem Biophys Res Commun 30:107(2):584-7 (1982); Potter et al., “Enhancer-Dependent Expression of Human Kappa Immunoglobulin Genes Introduced into Mouse pre-B Lymphocytes by Electroporation,” Proc. Natl. Acad. Sci. USA 81:7161-65 (1984, which are hereby incorporated by reference in their entirety) and polyethylene glycol (PEG) mediated DNA uptake, Sambrook et al., Molecular Cloning: A Laboratory Manual, Chap. 16, Second Edition, Cold Springs Laboratory, Cold Springs Harbor, N.Y. (1989), which is hereby incorporated by reference in its entirety). During electroporation, the DNA is introduced into the cell by means of a reversible change in the permeability of the cell membrane due to exposure to an electric field. PEG transformation introduces the DNA by changing the elasticity of the membranes. Unlike electroporation, PEG transformation does not require any special equipment and transformation efficiencies can be equally high. Another appropriate method of introducing the nucleic acid construct of the present invention into a host is fusion of protoplasts with other entities, either minicells, cells, lysosomes, or other fusible lipid-surfaced bodies that contain the chimeric gene (Fraley, et al., Proc Natl Acad Sci USA 79:1859-63 (1982), which is hereby incorporated by reference in its entirety).

Stable transformants are preferable for the methods of the present invention, which can be achieved by using variations of the methods above as describe in Sambrook et al., Molecular Cloning: A Laboratory Manual, Chap. 16, Second Edition, Cold Springs Laboratory, Cold Springs Harbor, N.Y. (1989), Ausubel et al., “Short Protocols in Molecular Biology,” New York:Wiley (1999), and U.S. Pat. No. 4,237,224 to Cohen and Boyer, which are hereby incorporated by reference in their entirety.

Suitable tissue that may be treated according to this aspect of the present invention includes, for example, ischemic tissue and tissue located at a wound site.

HIF-1 and/or SDF-1 activity may also be regulated by administration of agents that interfere with HIF-1 and/or SDF-1 activity. Suitable agents according to this aspect of the present invention include, e.g., agents that disrupt or reduce transcription or translation; agents that interfere with active sites on HIF-1 and/or SDF-1 that are involved in stem/progenitor cell recruitment, for example, agents that interfere with SDF-1-CXCR4⁺ interaction; and agents that increase the rate of HIF-1 and/or SDF-1 degradation.

Suitable HIF-1 antagonists include, for example, 103D5R (Tan et al., “Identification of a Novel Small-molecule Inhibitor of the Hypoxia-inducible Factor 1 Pathway,” Cancer Res. 65(2):605-612 (2005), which is hereby incorporated by reference in its entirety), and NSC-134754 and NSC-643735 (Chau et al., “Identification of Novel Small Molecule Inhibitors of Hypoxia-inducible Factor-1 that Differentially Block Hypoxia-inducible Factor-1 Activity and Hypoxia-inducible Factor-1α Induction in Response to Hypoxic Stress and Growth Factors,” Cancer Res. 65(11):4918-4928 (2005), which is hereby incorporated by reference in its entirety). Suitable HIF mimetics/agonists include, for example, desferrioxamine (also called deferoxamine) and cobalt chloride (Wang & Semenza, “Desferrioxamine Induces Erythropoietin Gene Expression and Hypoxia-inducible Factor 1 DNA-binding Activity: Implications for Models of Hypoxia Signal Transduction,” Blood 82(12):3610-3615 (1993), which is hereby incorporated by reference in its entirety), and the HIF activator protein PR39 (Li et al., “PR39, a Peptide Regulator of Angiogenesis,” Nat. Med. 6:49-55 (2000), which is hereby incorporated by reference in its entirety).

Suitable antagonists that interfere with SDF-1-CXCR4 interaction include, for example, AMD3100 (Broxmeyer et al., “Rapid Mobilization of Murine and Human Hematopoietic Stem and Progenitor Cells with AMD3100, a CXCR4 Antagonist,” J. Exp. Med. 201(8): 1307-1318 (2005); Rubin et al., “A Small-molecule Antagonist of CXCR4 Inhibits Intracranial Growth of Primary Brain Tumors, Proc. Nat'l. Acad. Sci. U.S.A. 100(23):13513-13518(2003), which are hereby incorporated by reference in their entirety) and silencing RNA for SDF-1.

Suitable agents also include, for example, antibodies against SDF-1 and/or CXCR4. Suitable antibodies include, for example, commercially-available antibodies such as Clone 44716, R&D Systems (anti-CXCR4) and Clone 79014.111, R&D Systems (anti-SDF-1), and antibodies raised against SDF-1 and/or CXCR4; and includes both monoclonal and polyclonal antibodies.

Monoclonal and polyclonal antibodies against SDF-1 and/or CXCR4 that are capable of inhibiting their activity may be produced using techniques that are well-known in the art. Basically, monoclonal antibody production involves first obtaining immune cells (lymphocytes) from the spleen of a mammal (e.g., mouse) which has been previously immunized with the antigen of interest (i.e., SDF-1 and/or CXCR4) either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, “Continuous Culture of Fused Cells Secreting Antibody of Predefined Specificity,” Nature, 256:495-7 (1975), which is hereby incorporated by reference in its entirety.

Mammalian lymphocytes are immunized by in vivo immunization of the animal (e.g., a mouse) with SDF-1 and/or CXCR4. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (“PEG”) or other fusing agents (Milstein et al., “Derivation of Specific Antibody-Producing Tissue Culture and Tumor Lines by Cell Fusion,” Eur. J. Immunol. 6:511-19 (1976), which is hereby incorporated by reference in its entirety). This immortal cell line, which may be derived from cells of any mammalian species, including, but not limited to, mouse, rat, and human, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also well known. Typically, such antibodies can be raised by administering SDF-1 and/or CXCR4 subcutaneously to New Zealand white rabbits which have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbital 150 mg/Kg IV. This and other procedures for raising polyclonal antibodies are disclosed in E. Harlow, et. al., Editors, Antibodies: a Laboratory Manual (1988), which is hereby incorporated by reference in its entirety.

The present invention also relates to a method of modulating recruitment of stem cells and/or progenitor cells to a selected tissue site. This method involves controlling HIF-1 and/or SDF-1 activity at the selected tissue site to a level different than that otherwise present at the selected tissue site, under conditions effective to modulate recruitment of stem cells and/or progenitor cells to the selected tissue site.

Modulation according to this aspect of the present invention includes both up-modulation (increased recruitment of stem/progenitor cells) and down-modulation (decreased recruitment of stem/progenitor cells). Increased HIF-1 and/or SDF-1 activity increased recruitment of stem and/or progenitor cells; decreased HIF-1 and/or SDF-1 activity increases recruitment of stem and/or progenitor cells.

This aspect of the present invention may be carried out in vitro or in vivo.

HIF-1 activity and/or SDF-1 activity may be controlled using the methods of regulation described above.

Suitable tissue sites according to this aspect of the present invention include, for example, sites where there is ischemia, sites where there is damaged tissue, wound sites, and tumor sites.

Suitable tissue sites according to this aspect of the present invention also include sites of tissue susceptible to damage. For example, stem/progenitor cells may be recruited as a prophylactic to diseased coronary blood vessels prone to infarction.

This aspect of the present invention also contemplates recruitment to sites for tissue engineering. For example, stem/progenitor cells may be recruited to lay down a vascular foundation (see, e.g., Ceradini & Gurtner, “Homing to Hypoxia: HIF-1 as a Mediator of Progenitor Cell Recruitment to Injured Tissue,” TCM 15(2):57-63 (2005), which is hereby incorporated by reference in its entirety) at a site where a new organ is being engineered.

Suitable stem cells and/or progenitor cells according to this aspect of the present invention, include, for example, CXCR4⁺ cells, hematopoeitic stem/progenitor cells (Peled et al., “Dependence of Human Stem Cell Engraftment and Repopulation of NOD/SCID Mice on CXCR4,” Science 283:845-848 (1999), which is hereby incorporated by reference in its entirety), skeletal and smooth muscle stem/progenitor cells (Ratajczak et al., “Expression of Functional CXCR4 by Muscle Satellite Cells and Secretion of SDF-1 by Muscle-derived Fibroblasts is Associated with the Presence of Both Muscle Progenitors in Bone Marrow and Hematopoietic Stem/Progenitor Cells in Muscles,” Stem Cells 21:363-371 (2003), which is hereby incorporated by reference in its entirety), and neural stem/progenitor cells (Reiss et al., “Stromal Cell-derived Factor 1 is Secreted by Meningeal Cells and Acts as Chemotactic Factor on Neuronal Stem Cells of the Cerebellar External Granular Layer,” Neuroscience 115:295-305 (2002), which is hereby incorporated by reference in its entirety).

When the methods of the present invention are carried out in vivo, HIF-1 and/or SDF-1 may be increased by administering to a subject a compound as described above. These compounds can be administered orally, parenterally, for example, intradermally, subcutaneously, intravenously, intramuscularly, intraperitoneally, by intranasal instillation, or by application to mucous membranes, such as that of the nose, throat, and bronchial tubes. They may be administered alone or with suitable pharmaceutical carriers, and can be in solid or liquid form, such as tablets, capsules, powders, solutions, suspensions, or emulsions.

The active compounds may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The compounds of the present invention may be administered directly to the targeted tissue. Additionally and/or alternatively, the compounds may be administered to a non-targeted area along with one or more tissue-specific agents that facilitate migration of HIF-1/SDF-1 to the targeted tissue.

The present invention also relates to a method of treating cancer in a subject. This method involves limiting HIF-1 and/or SDF-1 activity in cancerous tumor tissue in the subject under conditions effective to treat the subject's cancer.

Similar to tissue injury, neoplastic states are often characterized by profound levels of hypoxia. Recent reports indicate that cancer cells share the CXCR4/SDF-1 axis, and describe its involvement in metastasis (Staller et al., “Chemokine Receptor CXCR4 Downregulated by von Hippel-Lindau Tumour Suppressor pVHL,” Nature 425:307-311 (2003); Muller et al., “Involvement of Chemokine Receptors in Breast Cancer Metastasis,” Nature 410:50-56 (2001), which are hereby incorporated by reference in their entirety). However, the potential role of HIF-1 and SDF-1 in tumor growth and survival have not been described.

The link between hypoxia, HIF-1, SDF-1, and stem/progenitor cell recruitment, disclosed herein, suggests that the tumor-associated microenvironment may function to continuously recruit circulating stem and progenitor cells, effectively “hijacking” the body's capacity for tissue regeneration. Indeed, HIF-1 overexpression is a negative prognostic indicator in many human cancers (Semenza, “Targeting HIF-1 for Cancer Therapy,” Nat. Rev. Cancer 3:721-732 (2003), which is hereby incorporated by reference in its entirety). This implies that efforts to decrease tumor vascularity (such as via anti-angiogenesis approaches) may be counterproductive, because they increase tumor hypoxia, potentially enhancing recruitment of circulating stem and progenitor cells, and enlisting host mechanisms for survival and growth. Limiting HIF-1 and/or SDF-1 activity in cancerous tumor tissue, therefore, may be used to limit tumor growth and regeneration.

Cancers that may be treated according to this aspect of the present invention include, for example, breast cancer, colon cancer, central nervous system cancer, leukemia, melanoma, lung cancer, ovarian cancer, prostate cancer, and renal cancer.

HIF-1 and/or SDF-1 activity may be limited according to this aspect of the present invention by the methods described above for down-regulating their activity. In aspects of the present invention involving administering tissue-specific agents, such agents include those specific for the type of cancer to be treated.

The present invention may be further illustrated by reference to the following examples.

### EXAMPLES

### Example 1

### Mouse Ischemia Model and Recruitment Experiments

One common theme in the protean pathways for which SDF-1 is believed to be essential is hypoxia (Hitchon et al., “Hypoxia-induced Production of Stromal Cell-derived Factor 1 (CXCL12) and Vascular Endothelial Growth Factor by Synovial Fibroblasts,” Arthritis Rheum. 46:2587-2597 (2002), which is hereby incorporated by reference in its entirety). Thus, whether localized differences in SDF-1 expression and progenitor cell trafficking could be explained by local differences in oxygen tension was examined.

An ischemia model using athymic nude mice (Jackson) was used as described previously (Tepper et al., “Human Endothelial Progenitor Cells from Type II Diabetics Exhibit Impaired Proliferation, Adhesion, and Incorporation into Vascular Structures,” Circulation 106:2781-2786 (2002), which is hereby incorporated by reference in its entirety) in full accordance with the New York University Institutional Animal Care and Use Committee. In this model, a peninsular shaped incision was made dividing epidermis, papillary and reticular dermis, subcutaneous connective tissue, and skeletal muscle (panniculus camosus) from the systemic circulation on all but a single side, generating a reproducible gradient of ischemia. Following surgery, animals received an intracardiac injection of DiI-labeled (dioctadecyl-tetramethylindo-carbocyanine perchlorate, Molecular Probes) EPCs (5×10⁵) either alone, preincubated with HLA-Class I antibody (control, Pharmingen), preincubated with neutralizing CXCR4 antibody (Clone 44716, R&D Systems), or mixed with free HLA-Class I or neutralizing SDF-1 antibody (Clone 79014.111, R&D Systems). Free antibodies (without cells) were readministered via intraperitoneal injection 24 hours following surgery (Peled et al., “Dependence of Human Stem Cell Engraftment and Repopulation of NOD/SCID Mice on CXCR4,” Science 283:845-848 (1999), which is hereby incorporated by reference in its entirety). Prior to sacrifice at 2 and 14 days postoperatively, a subset of animals were perfused with FITC-labeled Lycopersicon esculentum lectin (Vector) to stain the functional microvasculature. Harvested tissue was snap frozen in liquid nitrogen, sectioned, and either mounted in DAPI medium (Vector) and/or stained for immunofluorescence microscopy. For endothelial progenitor cell (EPC) recruitment experiments, animals were sacrificed on days 2 and 14 postoperatively, and the number of DiI-labeled EPCs from five non-consecutive sections of each tissue area (A-C, and non-ischemic) were quantified at 200× in three random fields by a blinded investigator. Functional capillary density was determined on day 14 by in situ staining with FITC-lectin (above) (Takahashi et al., “Ischemia- and Cytokine-induced Mobilization of Bone Marrow-derived Endothelial Progenitor Cells for Neovascularization,” Nat. Med. 5:434-438 (1999), which is hereby incorporated by reference in its entirety). The endothelial phenotype of lectin⁺ cells was confirmed by co-localization with DiI (human endothelial cells)/anti-CD31 -PE (mouse endothelial cells, BD Pharmingen) immunostaining. For SDF-1, HIF-1α, and CD31 detection (without EPC administration), 10 μm sections were incubated with FITC-conjugated anti-CD31, anti-mouse HIF-1α monoclonal antibody (Novus), and/or rabbit anti-mouse SDF-1 (e-Bioscience) primary antibody with subsequent detection of unconjugated primary antibodies using appropriate conjugated secondary antibodies (Alexa Fluor 594 or 488, Molecular Probes). The Hypoxyprobe-1 kit (pimonidazole hydrochloride, Chemicon) was utilized according to the manufacturer's protocol with the supplied monoclonal antibody and Alexa Fluor 488-conjugated secondary antibody used for tissue detection.

### Example 2

### In Situ Hybridization

Frozen and fixed tissue sections (10 μm) were washed in PBS containing 0.2% RNase Block (InnoGenex), treated with proteinase K (20 μg/mL) for 10 minutes at 37° C., and acetylated for 10 minutes at room temperature. Digoxigenin labeled cRNA probes were generated from bases 1090-1318 of the mouse SDF-1 cDNA sequence using a commercially available kit (Roche). Hybridization was performed at 60° C. for 16 hours with cRNA probe (200 ng/mL) in a commercially available hybridization buffer (InnoGenex). After post-hybridization washes, SDF-1 mRNA signal was detected with alkaline phosphatase coupled anti-digoxigenin antibodies (1:1000, Roche) overnight at 4° C. and developed with BM-Purple AP Substrate containing 1 mM Levamisole. Sense probes served as controls.

### Example 3

### Quantitative Real-time RT-PCR

Total RNA was extracted from cultured cells or homogenized tissue using the Tri-Reagent (Sigma), and purified using the RNA easy kit (Qiagen). The RNA PCR Core kit (Applied Biosystems) was used to construct cDNA, which was used as template in real time PCR (Cepheid Smartcycler, primers listed in Example 14) using the Platinum SYBR Green Supermix-UDG (Invitrogen). Relative quantification of PCR products was calculated after normalization to β-actin or glyceraldehyde-3-phosphate dehydrogenase. Results are representative of three independent experiments. Products were sequenced to confirm their identity.

### Example 4

### SDF-1 ELISA

ELISA was performed using the human or mouse SDF-1 Quantikine kit according to the manufacturer's protocol (R&D Systems). Cell culture supernatants and tissue homogenates after protein extraction with the TPER reagent (Pierce) were used following standardization of each sample by total protein content using the BCA Protein Assay Kit (Pierce). Results are representative of three independent experiments.

### Example 5

### Cell Culture

HUVECs (Clonetics) and HMEC-1 (CDC, Atlanta, Ga.) were cultivated in EGM-2 (Clonetics). Human EPCs were harvested from healthy donors as previously described (Tepper et al., “Human Endothelial Progenitor Cells from Type II Diabetics Exhibit Impaired Proliferation, Adhesion, and Incorporation into Vascular Structures,” Circulation 106:2781-2786 (2002); Kawamoto et al., “Therapeutic Potential of Ex Vivo Expanded Endothelial Progenitor Cells for Myocardial Ischemia,” Circulation 103:634-637 (2001), which are hereby incorporated by reference in their entirety) after obtaining informed consent in accordance with the New York University Institutional Review Board. Identification and estimation of EPC culture purity (90-95%) was determined by the percentage of cells that co-stain with FITC-labeled Ulex europaeus lectin I (UEA-1, Vector) and DiI-acLDL uptake, and confirmed by co-expression of CD31 (Tepper et al., “Human Endothelial Progenitor Cells from Type II Diabetics Exhibit Impaired Proliferation, Adhesion, and Incorporation into Vascular Structures,” Circulation 106:2781-2786 (2002); Dimmeler et al., “HMG-CoA Reductase Inhibitors (Statins) Increase Endothelial Progenitor Cells Via the PI 3-Kinase/Akt Pathway,” J. Clin. Invest. 108:391-397 (2001), which are hereby incorporated by reference in their entirety). Hypoxic culture conditions (1% O₂) were achieved in a custom designed hypoxic incubator using a continuous infusion of a pre-analyzed gas mixture (95% N₂, 5% CO₂). Media was serum starved (EBM-2/0.5% FBS) at least 8 hours prior to hypoxic culture to minimize the effects of growth factors in the expansion media.

### Example 6

### Chromatin Immunoprecipitation Assays (ChIP)

HUVECs were grown to 90% confluence, exposed to normoxia or hypoxia, and ChIP was performed using a commercially available kit according to the manufacturers protocol (Upstate). Antibodies to HIF-1α or mouse IgG (Pharmingen) were used to immunoprecipitate DNA fragments which were analyzed by real time quantitative PCR using primers specific for the SDF1 promoter as described in Example 14, β-actin, or glyceraldehyde-3-phosphate dehydrogenase. Products were sequenced to confirm their identity.

### Example 7

### Adhesion Assays

Confluent HUVEC monolayers were subjected to normoxic or hypoxic culture for 6 hours. Human SDF-1αβ/PBSF (100 ng/ml, Sigma) was added where indicated. DiI-labeled EPCs (5×10⁴) in EBM/0.5% FBS were added to each preconditioned monolayer and pre-adhesion fluorescence was measured using a Cytofluor 2320 (Millipore). After 3 hours, nonadherent cells were washed away, and post-adhesion fluorescence was measured. The percentage of adherent

cells was calculated using the following formula:

$$\% cells bound = \frac{post \text{-} adhesion fluorescence - monolayer only}{pre \text{-} adhesion fluorescence - monolayer only} \times 100$$

Results are representative of three independent experiments.

### Example 8

### Migration Assays

Migration was studied using a modified transwell assay. EPCs (5×10⁴) were seeded onto ChemoTx filters (5.7 mm, 8 μm pore, Neuro Probe) in EBM/0.5% FBS. Recombinant human SDF-1αβ/PBSF (Sigma) and conditioned media (EBM/0.5% FBS) from HUVECs cultured in different oxygen tensions for 6 hours were then added to the lower chamber. Following the 6 hour migration period, nonmigrating cells were completely wiped from the top surface of the membrane. Migrating cells adherent to the undersurface of the filters were quantified using DAPI staining (Vector) with Kodak 1D software. Results are indicative of four independent experiments.

### Example 9

### Statistical Analysis

Data are expressed as mean ±SEM. Data were analyzed using unpaired two-tailed Student's t-test or ANOVA and post hoc Tukey's test for multiple pairwise comparisons. Probability values of P<0.05 were considered statistically significant.

### Example 10

### Tissue Perfusion and Oxygen Tension Measurements

The OXYLAB pO₂ Tissue Oxygenation and Temperature Monitor (Oxford Optronix) was used for all oxygen tension measurements (Braun et al., “Comparison of Tumor and Normal Tissue Oxygen Tension Measurements Using OxyLite or Microelectrodes in Rodents,” Am. J. Physiol. Heart Circ. Physiol. 280:H2533-2544 (2001), which is hereby incorporated by reference in its entirety). An optical fiber probe (100 μm radius) matched with a thermocoupler was directly inserted into tissue, allowing for continuous temperature-compensated oxygen tension measurements (10 values/s). The probe was positioned at each reference point (p1-4) for 60 seconds, generating an average of 600 values per trial. Perfusion was measured with color laser Doppler (Moor Instruments). Relative blood flow was calculated as previously described (Aicher et al., “Essential Role of Endothelial Nitric Oxide Synthase for Mobilization of Stem and Progenitor Cells,” Nat. Med. 9:1370-1376 (2003); Couffinhal et al, “Mouse Model of Angiogenesis,” Am. J. Pathol. 152:1667-1679 (1998), which are hereby incorporated by reference in their entirety). For measurements of bone marrow oxygen tension, the patellar ligament was exposed and divided through a small incision, providing direct access to the distal femoral articular structure. An 18-gauge needle was used to drill through the outer cortical bone and epiphysis, providing a path for insertion of the oxygen tension probe directly in the marrow cavity of the femoral shaft.

### Example 11

### Generation of Reporter Constructs and Reporter Assays

The 1.4 kb 5′ SDF1 promoter region was cloned into the luciferase reporter vector pGL3-Basic (Promega) generating pGL.SDF1.full. 5′ deletion constructs were generated from pGL.SDF1.full using unique internal restriction sites SmaI/SacI, and XhoI. HBS-only contructs (HBS1, HBS2, or both) were generated by PCR and cloned into pGL3-Promoter (Promega). HBS1 was mutated using the mega-primer method (primers listed below) (Kammann et al., “Rapid Insertional Mutagenesis of DNA by Polymerase Chain Reaction (PCR),” Nucleic Acids Res. 17:5404 (1989), which is hereby incorporated by reference in its entirety). All constructs were sequenced to confirm their identity. Reporter plasmids were co-transfected with a constitutively expressed Renilla luciferase construct (pHRL-TK, Promega) into HMECs using the Genejammer reagent (Stratagene). 48 hours after transfection, cells were incubated in hypoxia or normoxia with and without 0.1 mM cobalt chloride (CoCl₂) for 12 hours. Luciferase activity was determined using the Dual Luciferase System (Promega) and data were normalized to Renilla luciferase expression from at least four independent experiments. Co-transfection experiments using plasmids for oxygen dependent HIF-1α (HIF-1.ΔODD) and the HIF-1α dominant negative (HIF-1.DN) were compared to control transfections using the appropriate empty vector (pcDNA3.1 and pCEP4, respectively) for each construct.

### Example 12

### HIF-1α RNA Interference

HIF-1α siRNA (Berra et al, “HIF Prolyl-hydroxylase 2 is the Key Oxygen Sensor Setting Low Steady-state Levels of HIF-1α in Normoxia,” Embo. J. 22:4082-4090 (2003), which is hereby incorporated by reference in its entirety) (synthesized by Qiagen) was transfected into HUVECs using RNAiFect (Qiagen) in parallel with Lamin A/C and FITC-labeled control siRNAs according to the manufacturer=3 s protocol. Cells were treated with normoxic or hypoxic conditions starting 24 hours following transfection. Gene expression and adhesion assays were performed at 48 hours.

### Example 13

### Flow Cytometry

Cells (10⁵) were blocked in PBS-10% FBS and stained for 30 minutes at 4° C. with primary antibodies for CXCR4 (Pharmingen, clone 12G5), KDR/FLK-1 (Sigma), SDF-1 (R&D Systems), or CD31 (Pharmingen) followed by detection with conjugated secondary antibody (Alexafluor 488, Molecular Probes). Samples were analyzed on a FACStar flow cytometer (Becton Dickinson).

### Example 14

### Primers

The following primers were used for quantitative real-time RT-PCR:

| | |
|---|---|
| mouse β-actin (forward): | |
| 5′-ACCAACTGGGACGATATGGAGAAGA-3′ | (SEQ ID NO: 1) |
| mouse β-actin (reverse): | |
| 5′-TACGACCAGAGGCATACAGGGACAA-3′ | (SEQ ID NO: 2) |
| mouse SDF-1 (forward): | |
| 5′-CAGCCGTGCAACAATCTGAAG-3′ | (SEQ ID NO: 3) |
| mouse SDF-1 (reverse): | |
| 5′-CTGCATCAGTGACGGTAAACC-3′ | (SEQ ID NO: 4) |
| mouse GAPDH (forward): | |
| 5′-AACATCATCCCTGCATCCAC-3′ | (SEQ ID NO: 5) |
| mouse GAPDH (reverse): | |
| 5′-CCCTGTTGCTGTAGCCGTAT-3′ | (SEQ ID NO: 6) |
| human β-actin (forward): | |
| 5′-GCCGATCCACACGGAGTACT-3′ | (SEQ ID NO: 7) |
| human β-actin (reverse): | |
| 5′-CTGGCACCCAGCACAATG-3′ | (SEQ ID NO: 8) |
| human SDF-1 (forward): | |
| 5′-GTGTCACTGGCGACACGTAG-3′ | (SEQ ID NO: 9) |
| human SDF-1 (reverse): | |
| 5′-TCCCATCCCACAGAGAGAAG-3′ | (SEQ ID NO: 10) |
| human HIF-1α (forward): | |
| 5′-CCATTAGAAAGCAGTTCCGC-3′ | (SEQ ID NO: 11) |
| human HIF-1α (reverse): | |
| 5′-TGGGTAGGAGATGGAGATGC-3′ | (SEQ ID NO: 12) |
| human GAPDH (forward): | |
| 5′-AACATCATCCCTGCCTCTAC-3′ | (SEQ ID NO: 13) |
| human GAPDH (reverse): | |
| 5′-CCCTGTTGCTGTAGCCAAAT-3′ | (SEQ ID NO: 14) |

The following primers were used for chromatin immunoprecipitation (ChIP):

human SDF1 promoter (ChIP forward):
5′-TCTAACGGCCAAAGTGGTTT-3′ (SEQ ID NO: 15)
human SDF1 promoter (ChIP reverse):
5′-GCCACCTCTCTGTGTCCTTC-3′ (SEQ ID NO: 16)

The following primers were used for SDF1 promoter cloning and mutagenesis:

SDF1.full (forward):

(SEQ ID NO: 17)

5′-CGCGGATCCGGCCCACAGCCATCTAACGGC-3′
SDF1.full (reverse):

(SEQ ID NO: 18)

5′-CCGGAATTCGCAATGCGGCTGACGGAGAGTGA-3′
HBS1 (forward):

(SEQ ID NO: 19)

5′-GCGGGTACCCTAATGCAGCCGCTGACC-3′
HBS1 (reverse):

(SEQ ID NO: 20)

5′-GCGGCTAGCCTTTGGGCCTCGCTTTGT-3′
HBS2 (forward):

(SEQ ID NO: 21)

5′-GCGGGTACCCTGCTTGTCAGACACGATGC-3′
HBS2 (reverse):

(SEQ ID NO: 22)

5′-GCGGCTAGCCCTCAGTTTCCTCGCCTGTA-3′
HBS1.mut (reverse):

(SEQ ID NO: 23)

5′-CCTGCCCTGGGGA [symbol] CCCTGTCCCTG-3′

### Example 15

### In Vivo Investigation of Hypoxia and SDF-1 Regulation (Soft Tissue Ischemia Model)

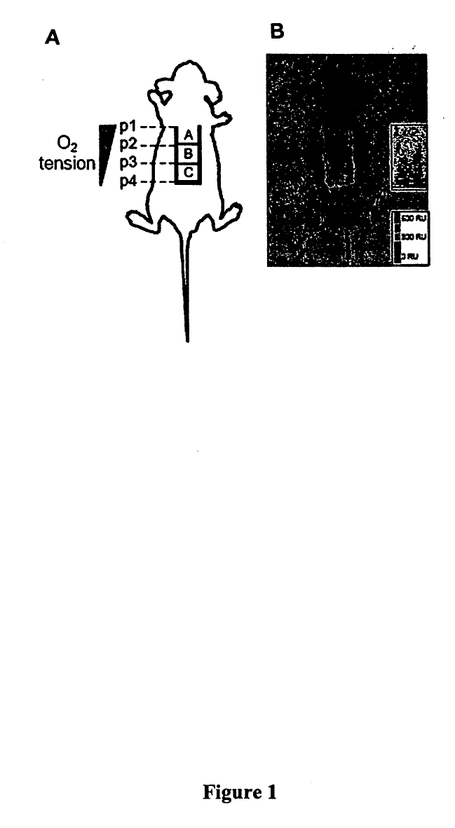

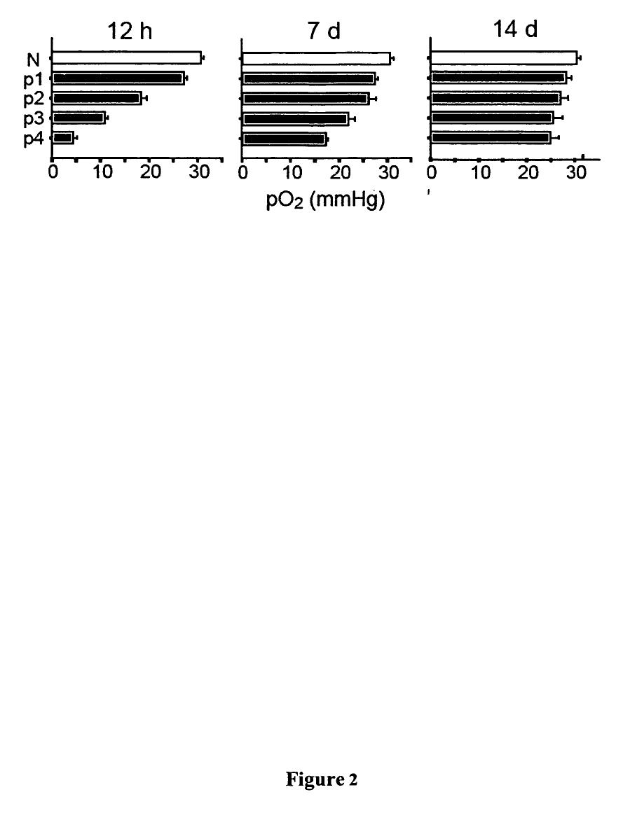

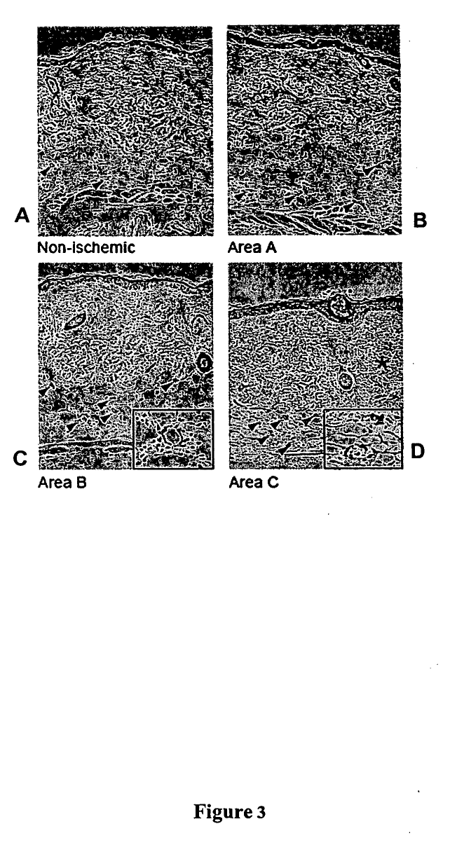

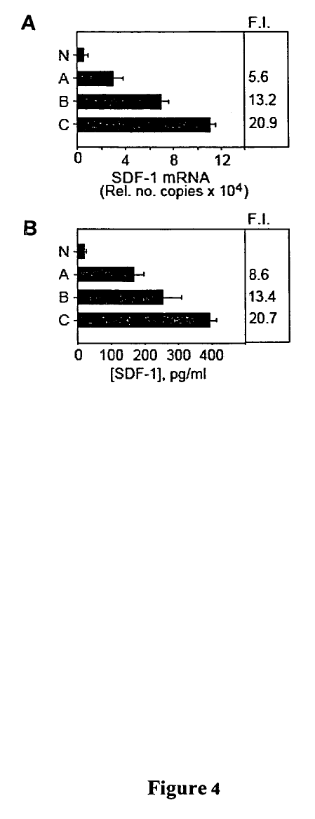

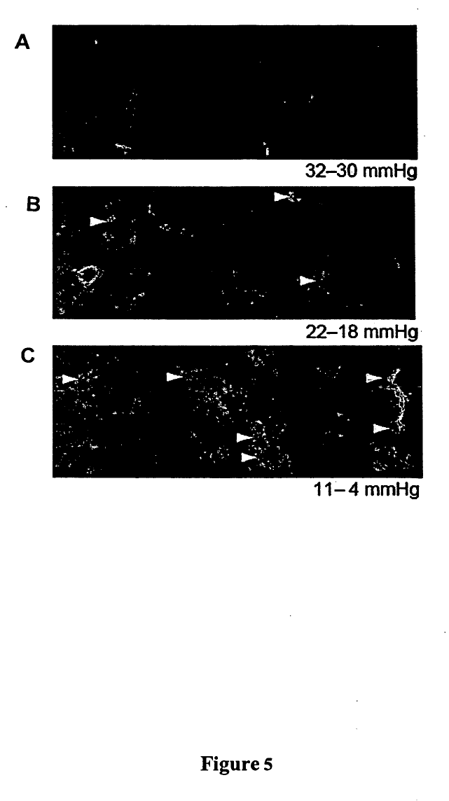

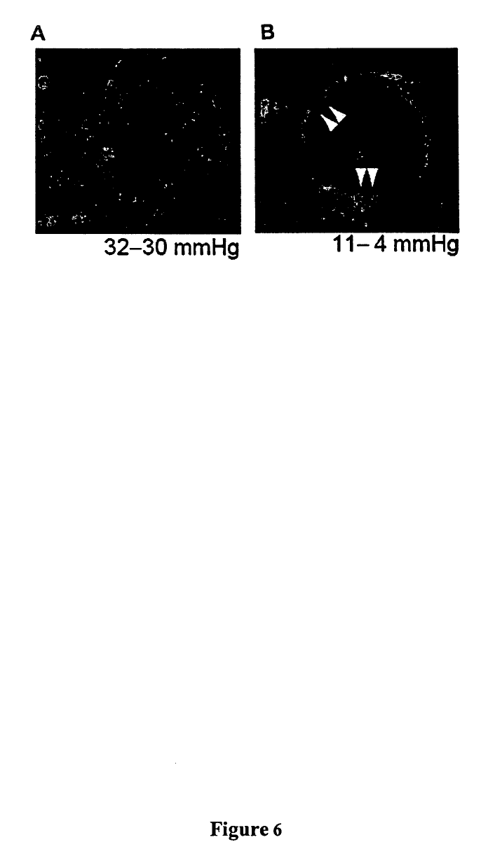

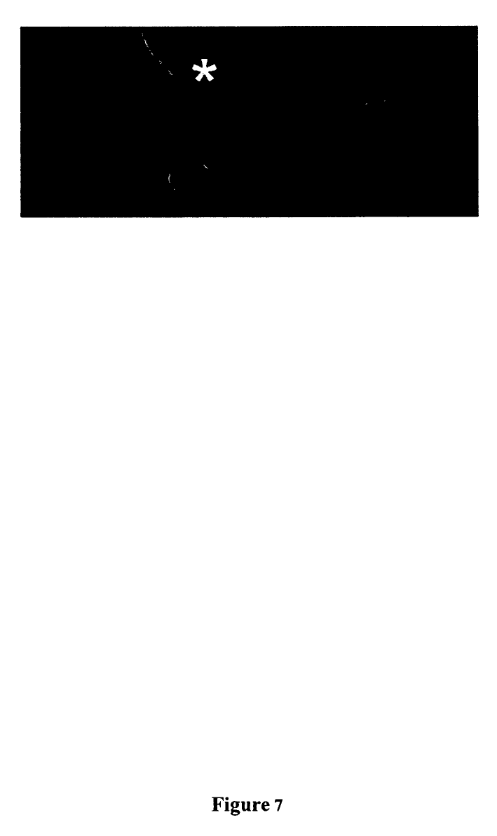

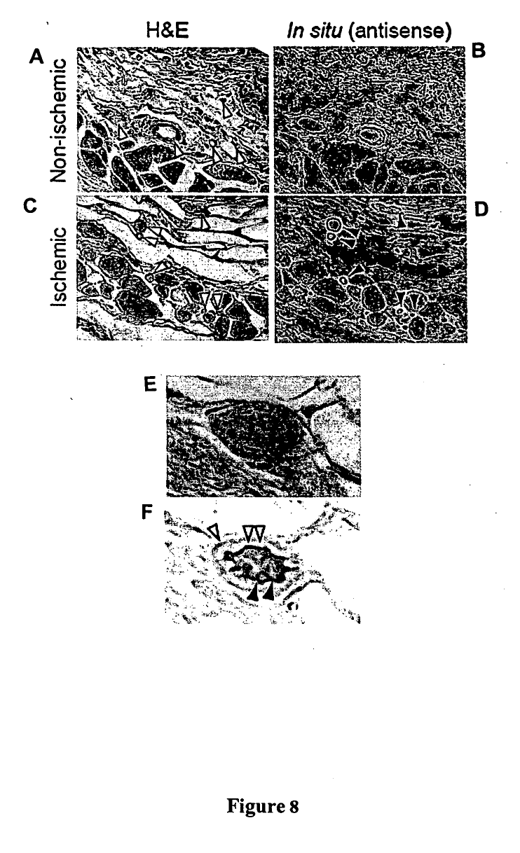

The potential impact of reduced oxygen tension on SDF-1 regulation in vivo was investigated using a soft tissue ischemia model in athymic nude mice (Tepper et al., “Human Endothelial Progenitor Cells from Type II Diabetics Exhibit Impaired Proliferation, Adhesion, and Incorporation into Vascular Structures,” Circulation 106:2781-2786 (2002), which is hereby incorporated by reference in its entirety) as shown in FIG. 1. Direct measurement of oxygen tension allowed for the definition of three discrete tissue segments (areas A, B, and C, as illustrated in FIG. 1A) with increasingly ischemic microenvironments (see Example 1). In this model, the nadir of tissue oxygenation occurs in the first 12 hours followed by a progressive increase in oxygen tension over the course of 14 days, as shown in FIG. 2, with a mild inflammatory response, as shown in FIG. 3. A dramatic increase in SDF-1 mRNA in ischemic tissue was observed 6 hours after surgery, as shown in FIG. 4A, that was directly proportional to reduced tissue oxygen tension levels, resulting in a similar increase in SDF-1 protein expression, as shown in FIG. 4B. Further, immunohistochemistry revealed no detectable SDF-1 expression in non-ischemic tissue, as shown in FIGS. 5A and 6A. In contrast, SDF-1 expression was abundant in ischemic tissue in a vascular and perivascular distribution, both in the endothelial cells and lining the vascular lumen, as shown in FIGS. 5B-C and FIG. 6B. Co-localization of monoclonal antibody CD31 and SDF-1 immunostaining, as shown in FIG. 7, suggests that endothelial cells are a source of SDF-1 expression in ischemic tissue, which was confirmed by in situ hybridization, as shown in FIGS. 8A-F.

This soft tissue ischemia nude mice model demonstrates that SDF-1 expression is directly proportional to reduced tissue oxygen tension in vivo.

### Example 16

### In Vitro Examination of SDF-1 Expression in Endothelial Cells

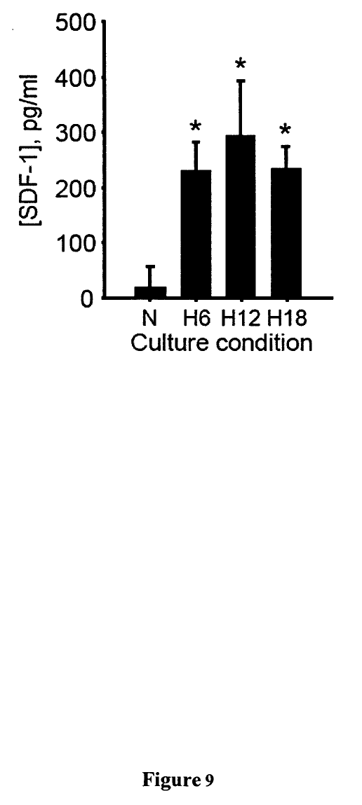

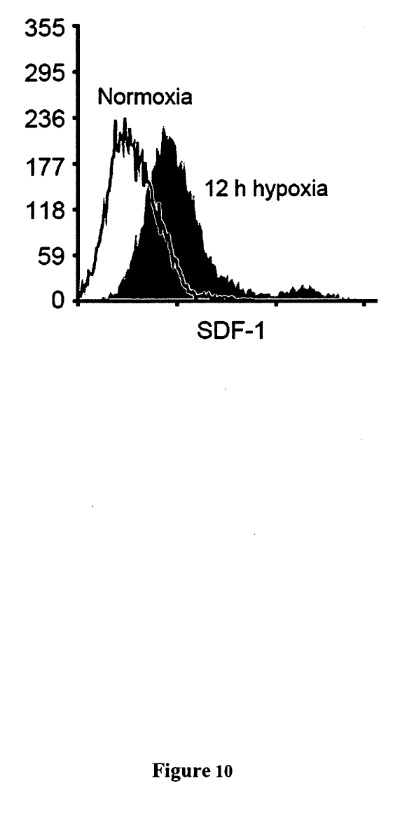

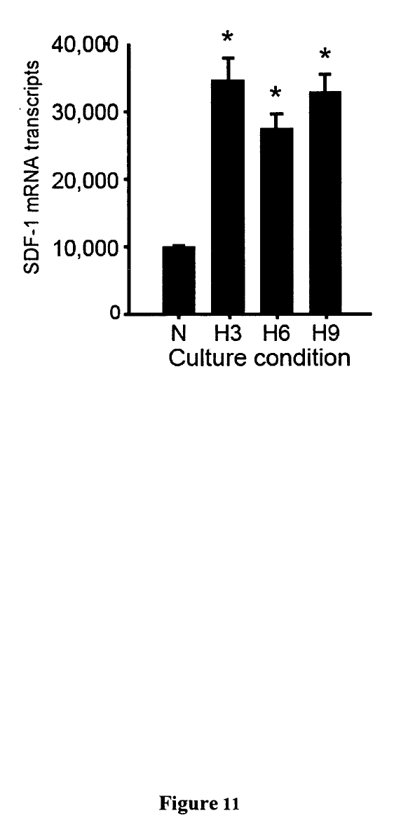

In order to study the molecular mechanism of hypoxia-induced SDF-1 expression, human endothelial cells (HUVECs) were examined in vitro. Oxygen levels in tissue culture were maintained at 1% (7.2 mmHg), which corresponded to the most ischemic tissue area in the animal model (Area C, FIG. 1A). Secreted SDF-1 was elevated seven-fold in culture media after 6 hours of hypoxia, reaching maximum levels (nine-fold) by 12 hours, as shown in FIG. 9. Flow cytometry revealed an increase in endothelial cell surface-bound SDF-1, as shown in FIG. 10, likely through binding to heparan sulfates on the endothelial surface (Amara et al., “Stromal Cell-derived Factor-1α Associates with Heparan Sulfates through the First β-Strand of the Chemokine,” J. Biol. Chem. 274:23916-23925 (1999), which is hereby incorporated by reference in its entirety). Quantitative real time RT-PCR demonstrated a three-fold increase in the relative number of SDF-1 mRNA transcripts after 3, 6, and 9 hours of hypoxia, as shown in FIG. 11. These findings suggest that hypoxia specific transcriptional elements are a primary control mechanism for SDF-1 expression.

### Example 17

### Identification of HIF-1 Binding Site on SDF-1

Hypoxia-inducible factor (HIF) is the central mediator of the cellular response to hypoxia, regulating over 60 genes that affect cell survival and metabolism in adverse conditions (Semenza, “Targeting HIF-1 for Cancer Therapy,” Nat. Rev. Cancer 3:721-732 (2003), which is hereby incorporated by reference in its entirety). It has never been demonstrated to directly regulate a member of the chemokine family, such as SDF-1.

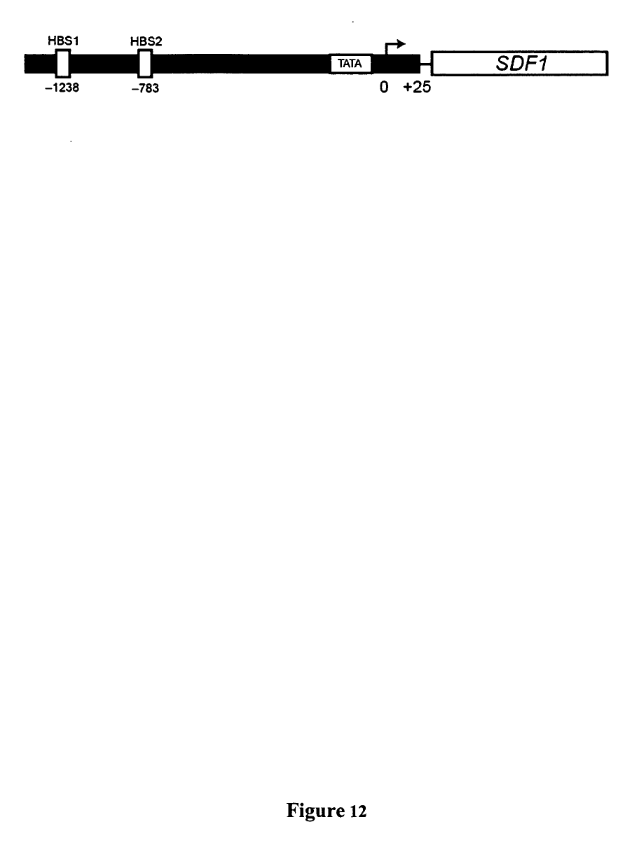

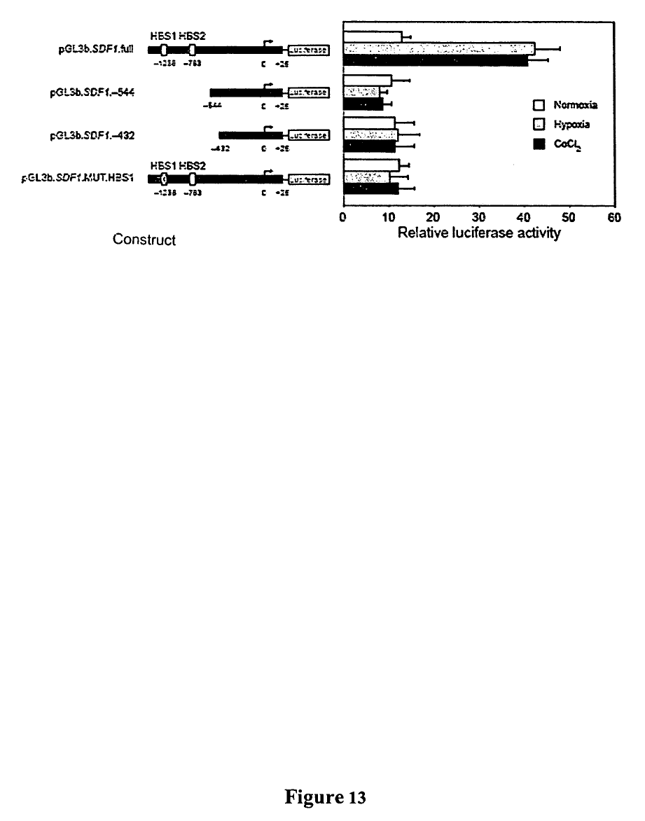

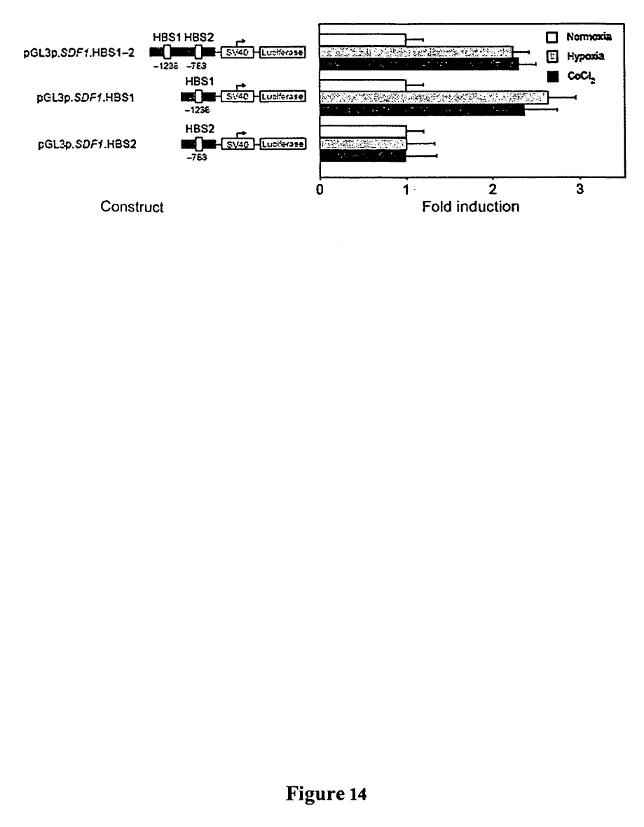

Analysis of the 5′ flanking region of the human SDF1 gene revealed two potential HIF-1 binding sites based on previously published consensus sequences (A/GCGTG) at −1238 (HBS1) and −783 (HBS2), as shown in FIG. 12. The 5′ 1.4 kb putative SDF1 promoter sequence was cloned from human genomic DNA into a luciferase reporter vector (pGL3b.SDF1.full) as described in Example 11. Transient transfection of this construct into human microvascular endothelial cells (HMEC-1) revealed that SDF1-specific luciferase expression increased four-fold after exposure to both hypoxic conditions and 0.1 mM cobalt chloride (CoCl₂, a known HIF-1 mimetic), as shown in FIG. 13. Serial 5′ deletion analysis of the SDF1 promoter revealed that removal of the putative HIF-1 binding sites abolished hypoxia and CoCl₂-inducible gene expression, while site-directed mutagenesis of HBS1 in the full length construct produced a similar effect, as shown in FIG. 13. Insertion of HBS1 and HBS2 upstream from a minimal SV40 promoter confirmed that HBS1 was sufficient to confer hypoxia specific gene expression, as shown in FIG. 14.

### Example 18

### Specificity of HIF-1 Activation of SDF-1 Transcription

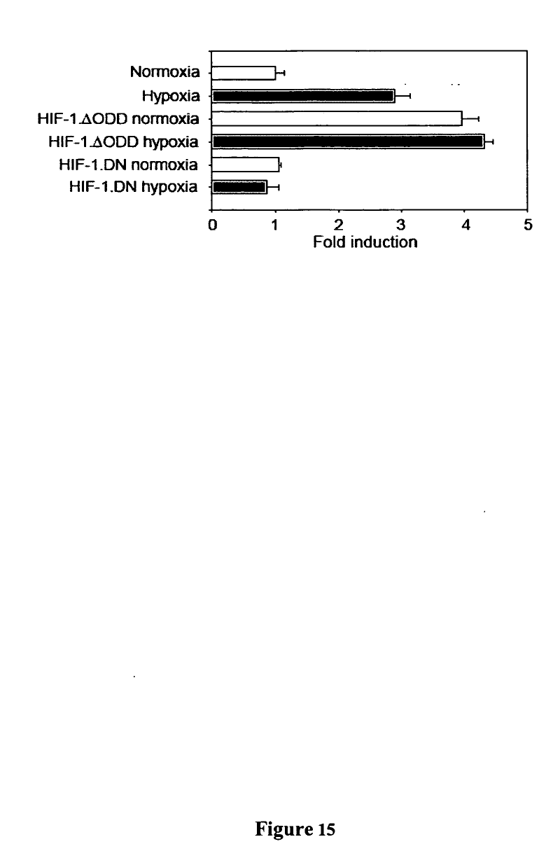

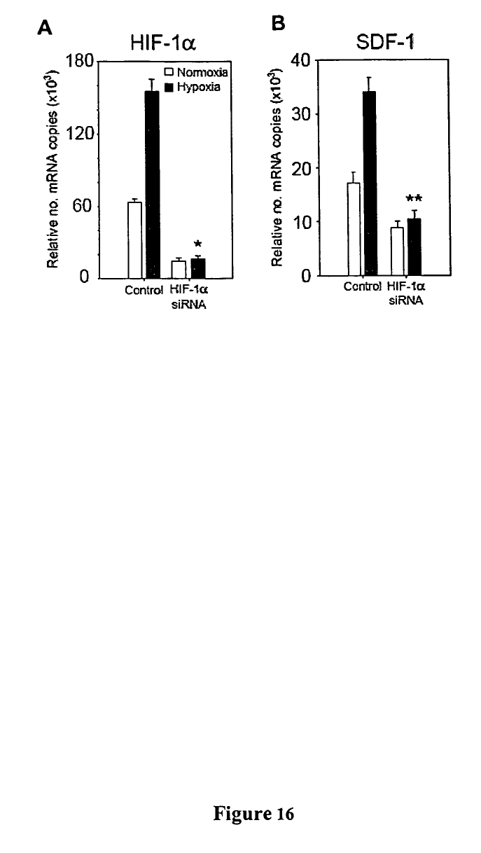

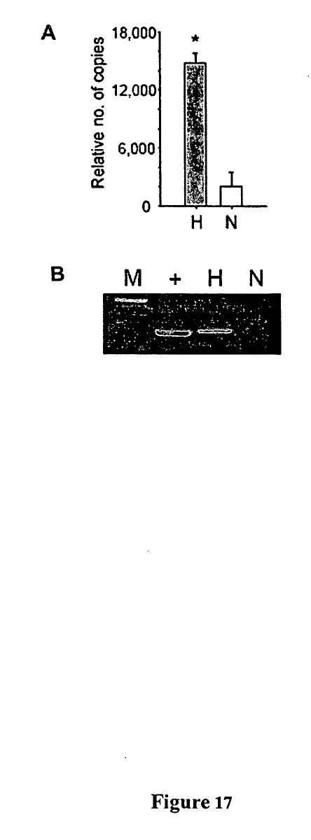

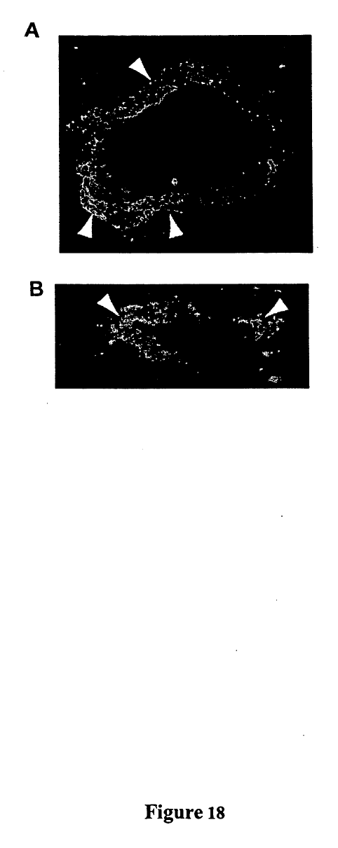

To demonstrate the specificity of HIF-1 activation of SDF-1 transcription, HMECs were co-transfected with the full length SDF1 reporter and constructs encoding either a constitutively active, oxygen independent HIF-1α mutant (HIF-1.ΔODD, which lacks the oxygen dependent degradation domain (Huang et al., “Regulation of Hypoxia-inducible Factor 1α is Mediated by an O₂-dependent Degradation Domain Via The Ubiquitin-proteasome Pathway,” Proc. Natl. Acad. Sci. U.S.A. 95:7987-7992 (1998), which is hereby incorporated by reference in its entirety)) or a constitutively expressed HIF-1α dominant negative (HIF-1.DN, which lacks the basic DNA binding and carboxy-terminal transactivation domains (Forsythe et al., “Activation of Vascular Endothelial Growth Factor Gene Transcription by Hypoxia-inducible Factor 1,” Mol. Cell. Biol. 16:4604-4613 (1996), which is hereby incorporated by reference in its entirety)). Co-expression of HIF-1.ΔODD induced a four-fold transcriptional activation of the SDF1 promoter in both normoxia and hypoxia, while co-expression of HIF-1.DN abolished hypoxia-responsive expression, as shown in FIG. 15. Furthermore, selective gene silencing of HIF-1α using specific siRNA (see Example 12) abolished HIF-1α expression under hypoxic conditions, as shown in FIG. 16A, and blocked hypoxia-specific SDF-1 expression compared to control siRNA, as shown in FIG. 16B. Chromatin immunoprecipitation with HIF-1α antibodies (FIG. 17B) and subsequent quantitative real time PCR demonstrated that HIF-1α is directly bound by the SDF1 promoter over seven-fold more in hypoxic conditions, as shown in FIG. 17A. In vivo, HIF-1α and SDF-1 co-localized in approximately 76% of ischemic endothelial cells, mainly those lining small caliber arterioles, venules and capillaries, as shown in FIGS. 18A-B. These data demonstrate that SDF-1 expression is transcriptionally activated by oxygen-dependent stabilization of HIF-1α, and is the first evidence of direct chemokine regulation by HIF-1.

### Example 19

### In Vitro Adhesion Assays

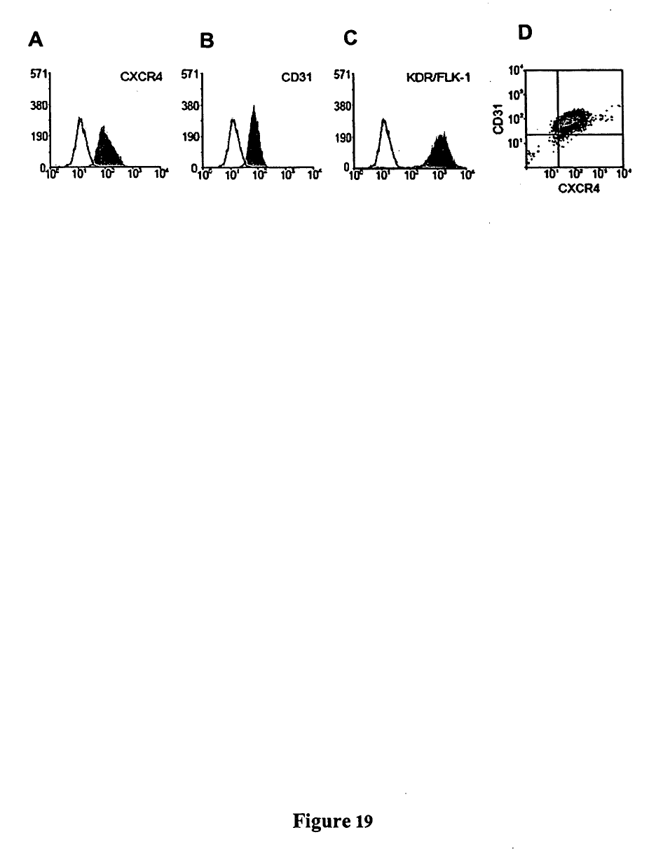

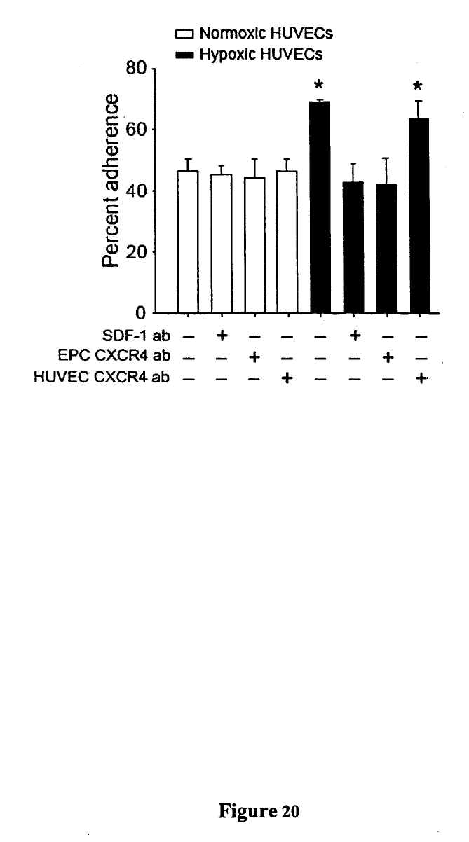

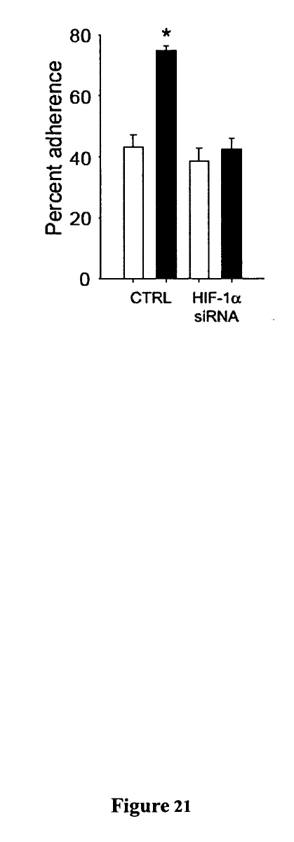

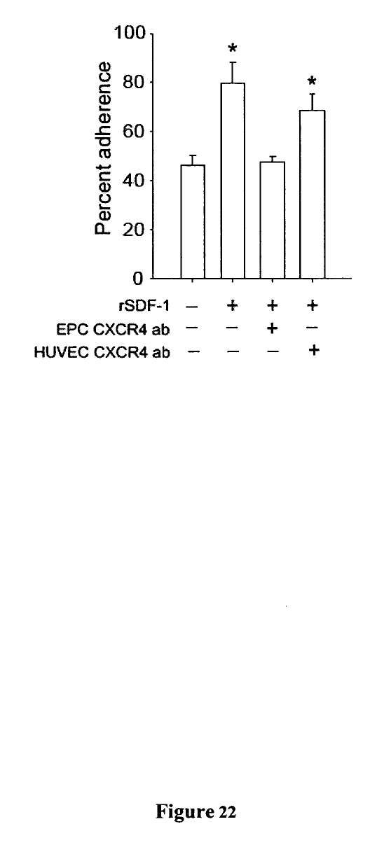

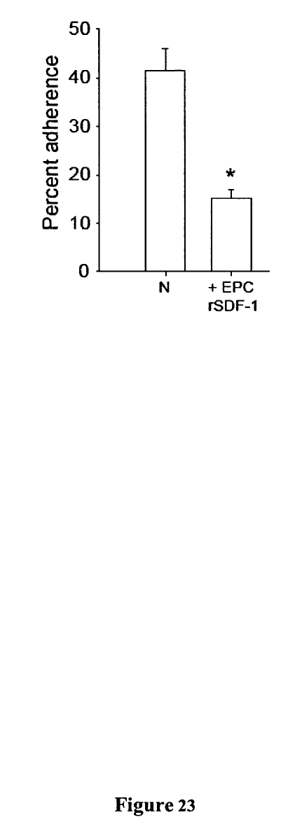

The functional significance of HIF-1 induced SDF-1 expression in endothelial cells was examined using adhesion assays in vitro. Prior studies have revealed that chemokines enhance integrin-mediated adhesion of circulating leukocytes to endothelial monolayers (Peled et al., “The Chemokine SDF-1 Stimulates Integrin-mediated Arrest of CD34⁺ Cells on Vascular Endothelium Under Shear Flow,” J. Clin. Invest. 104:1199-1211 (1999); Campbell et al., “Chemokines and the Arrest of Lymphocytes Rolling under Flow Conditions,” Science 279:381-384 (1998), which are hereby incorporated by reference in their entirety). CXCR4 is known to be highly expressed on a multitude of putative stem and progenitor cells, including hematopoietic, skeletal and smooth muscle, neural, and endothelial precursors (Peled et al., “Dependence of Human Stem Cell Engraftment and Repopulation of NOD/SCID Mice on CXCR4,” Science 283:845-848 (1999); Yamaguchi et al., “Stromal Cell-derived Factor-1 Effects on Ex Vivo Expanded Endothelial Progenitor Cell Recruitment for Ischemic Neovascularization,” Circulation 107:1322-1328 (2003); Reiss et al., “Stromal Cell-derived Factor 1 is Secreted by Meningeal Cells and Acts as Chemotactic Factor on Neuronal Stem Cells of the Cerebellar External Granular Layer,” Neuroscience 115:295-305 (2002); Ratajczak et al., “Expression of Functional CXCR4 by Muscle Satellite Cells and Secretion of SDF-1 by Muscle-derived Fibroblasts is Associated with the Presence of Both Muscle Progenitors in Bone Marrow and Hematopoietic Stem/Progenitor Cells in Muscles,” Stem Cells 21:363-371 (2003), which are hereby incorporated by reference in their entirety). Endothelial progenitor cells (EPCs) were examined as described in this Example, because ischemia-induced neovascularization is essential for tissue regeneration (Kawamoto et al., “Therapeutic Potential of Ex Vivo Expanded Endothelial Progenitor Cells for Myocardial Ischemia,” Circulation 103:634-637 (2001), which is hereby incorporated by reference in its entirety), and these cells are the most widely studied vascular progenitor (Yamaguchi et al., “Stromal Cell-derived Factor-1 Effects on Ex Vivo Expanded Endothelial Progenitor Cell Recruitment for Ischemic Neovascularization,” Circulation 107:1322-1328 (2003); Asahara et al., “Isolation of Putative Progenitor Endothelial Cells for Angiogenesis,” Science 275:964-967 (1997); Hill et al., “Circulating Endothelial Progenitor Cells, Vascular Function, and Cardiovascular Risk,” N. Engl. J. Med. 348:593-600 (2003), which are hereby incorporated by reference in their entirety). EPCs are known to highly express CXCR4 as well as CD31 and KDR/FLK-1, with >94% co-expressing CD31/CXCR4, as shown in FIG. 19A (CSCR4), FIG. 19B (CD31), FIG. 19C (KDR/FLK-1), and FIG. 19D (CD31/CXCR4). As shown in FIG. 20, HUVEC monolayers preconditioned in hypoxia for 6 hours were found to adhere a greater number of CXCR4⁺ EPCs than monolayers cultivated in normal oxygen tension (69.0%±0.52 versus 46.4%±2.3, P<0.001), an effect that could be abolished by antibody blockade of SDF-1/CXCR4 interactions. Silencing of HIF-1α expression in endothelial cells with siRNA also abolished the hypoxia-specific increase in adhesion, as shown in FIG. 21. Increased adhesion of CXCR4⁺ EPCs to normoxic HUVEC monolayers could be reproduced by pre-coating monolayers for 20 minutes with recombinant SDF-1, as shown in FIG. 22. As shown in FIG. 23, preincubation of CXCR4⁺ EPCs with soluble SDF-1 for 20 minutes prior to assay actually reduced adhesion below baseline (41.6%±4.5 vs. 15.1%±1.74, P<0.005), presumably due to a reduction in available cell surface receptors because of rapid CXCR4 internalization (Signoret et al., “Phorbol Esters and SDF-1 Induce Rapid Endocytosis and Down Modulation of the Chemokine Receptor CXCR4,” J. Cell Biol. 139:651-664 (1997), which is hereby incorporated by reference in its entirety). This suggests that circulating SDF-1 may act to desensitize stem and progenitor cells in the bone marrow, increasing their likelihood of mobilization (Sweeney et al., “Sulfated Polysaccharides Increase Plasma Levels of SDF-1 in Monkeys and Mice: Involvement in Mobilization of Stem/Progenitor Cells,” Blood 99:44-51 (2002), which is hereby incorporated by reference in its entirety), whereas SDF-1 immobilized on and around ischemic blood vessels facilitates tissue specific adhesion and localization.

### Example 20

### In Vitro Migration Assays

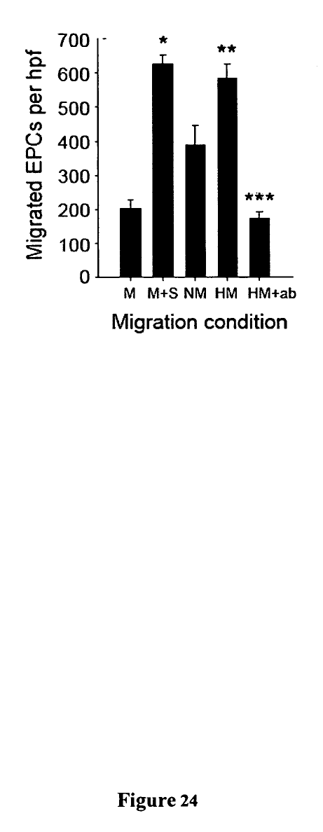

SDF-1 is also known to mediate the mobilization and migration of bone marrow derived stem and progenitor cells in vivo (Sweeney et al., “Sulfated Polysaccharides Increase Plasma Levels of SDF-1 in Monkeys and Mice: Involvement in Mobilization of Stem/Progenitor Cells,” Blood 99:44-51 (2002); Hattori et al., “Plasma Elevation of Stromal Cell-derived Factor-1 Induces Mobilization of Mature and Immature Hematopoietic Progenitor and Stem Cells,” Blood 97:3354-3360 (2001), which are hereby incorporated by reference in their entirety). As shown in FIG. 24, it was found that conditioned media from HUVECs cultivated in hypoxic conditions enhanced EPC migration when compared to media from HUVECs grown in normal oxygen tensions (585±21 cells/hpf (HM) versus 389±18.25 cells/hpf (NM); P<0.001). This increase in migration could be blocked by preincubating EPCs with neutralizing antibodies to CXCR4 (585±21 cells/hpf (HM) versus 175±9.8 cells/hpf (HM+ab); P<0.001) whereas isotype control antibodies had no effect on migration. Recombinant SDF-1 also induced robust migration in a dose dependent manner compared to control, with maximal response at 200 ng/ml (625±18 cells/hpf(M+S) versus 203±10 cells/hpf(M); P<0.001).

Examples 19 and 20 demonstrate that HIF-1 activated expression of SDF-1 mediates functional interactions between mature endothelium and circulating progenitor cells.

### Example 21

### In Vivo Investigation of Hypoxia and Circulating Progenitor Cell Recruitment (Soft Tissue Ischemia Model)

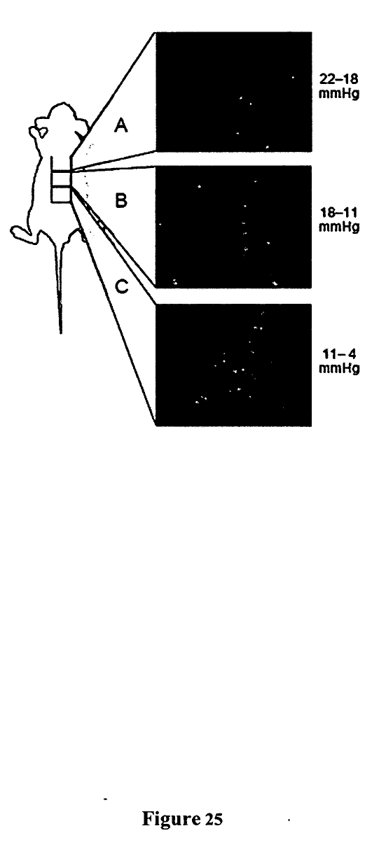

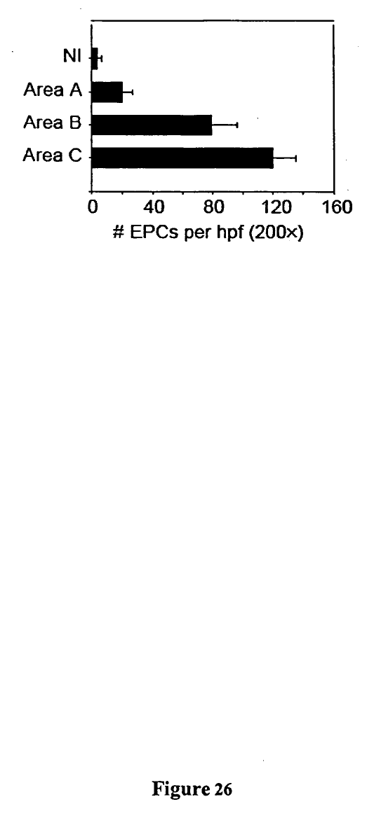

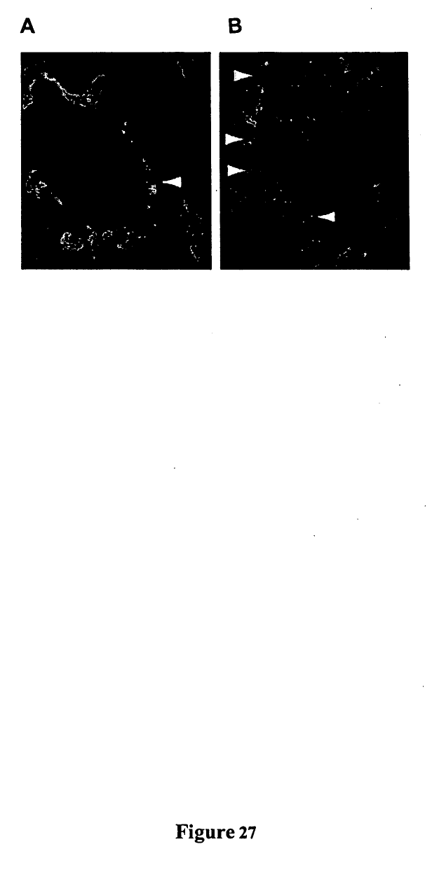

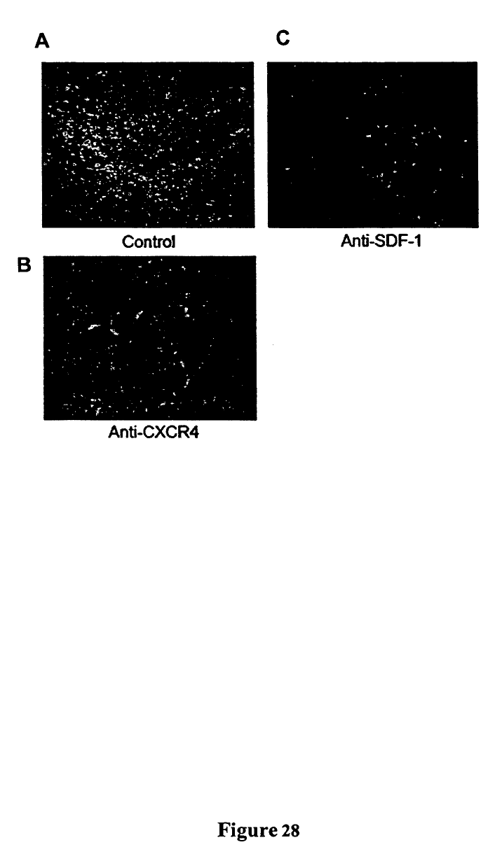

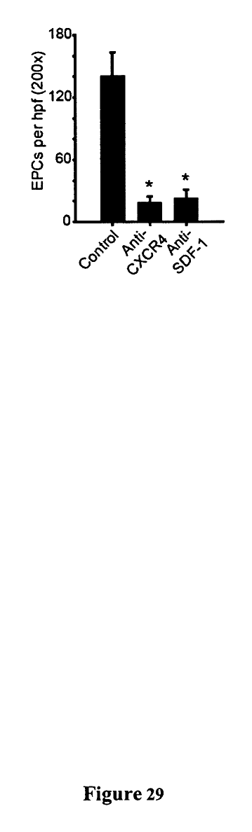

The mouse ischemia model was used to examine the influence of absolute tissue oxygen tension on circulating progenitor cell recruitment, and whether HIF-1 induced SDF-1 expression is necessary for this to occur. Previous studies have shown that EPCs localize to ischemic tissue and participate in tissue repair in several animal models (Yamaguchi et al., “Stromal Cell-derived Factor-1 Effects on Ex Vivo Expanded Endothelial Progenitor Cell Recruitment for Ischemic Neovascularization,” Circulation 107:1322-1328 (2003); Kawamoto et al., “Therapeutic Potential of Ex Vivo Expanded Endothelial Progenitor Cells for Myocardial Ischemia,” Circulation 103:634-637 (2001), which are hereby incorporated by reference in their entirety). As shown in FIGS. 25-26, it was found that the number of CXCR4⁺ EPCs homing to (day 2 postoperatively) and engrafting in (day 14 postoperatively) ischemic tissue following intravascular administration was directly proportional to reduced tissue oxygen tensions. The engraftment pattern was identical to SDF-1 expression levels demonstrated previously, and it was found that these cells rarely localized to vessels in non-ischemic tissue. In contrast, cells that homed to ischemic tissue were frequently found lining microvascular conduits, as shown in FIGS. 27A-B. To determine whether this was predominantly due to HIF-1-induced SDF-1 expression, specific antibody blockade of CXCR4/SDF-1 was utilized to examine the contribution of this pathway to progenitor cell localization in vivo, since blockade of HIF-1 would have many non-specific downstream effects (i.e. altered VEGF, iNOS, etc.), and both CXCR4 and SDF-1 null mice have an embryonic lethal phenotype (Nagasawa et al., “Defects of B-cell Lymphopoiesis and Bone-marrow Myelopoiesis in Mice Lacking the CXC Chemokine PBSF/SDF-1,” Nature 382:635-638 (1996); Zou et al., “Function of the Chemokine Receptor CXCR4 in Haematopoiesis and in Cerebellar Development,” Nature 393:595-599 (1998), which are hereby incorporated by reference in their entirety). As shown in FIGS. 28A-B and FIG. 29, ischemia-specific homing and engraftment of CXCR4⁺ EPCs was dramatically reduced by preincubating cells with neutralizing CXCR4 antibody prior to administration despite the persistence of a hypoxic microenvironment (n=4). In addition, intravenous administration of free neutralizing SDF-1 antibodies with progenitor cells reduced ischemia-specific engraftment to a similar degree, as shown in FIG. 28C and FIG. 29 (n=4).

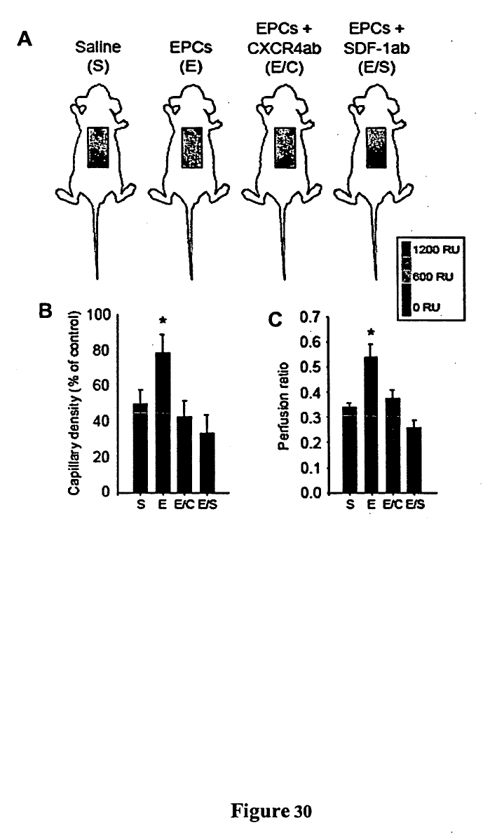

As shown in FIGS. 30A-C, functionally, EPC administration significantly improved ischemic tissue perfusion and capillary density after 14 days compared to control animals, an effect which could be abrogated by CXCR4 or SDF-1 blockade (n=4). Blockade of host derived SDF-1 (E/S) resulted in greater impairments in perfusion and capillary density at 14 days, likely by interfering with native mouse endothelial progenitor trafficking and neovascularization. Administration of CXCR4⁺ EPCs 7 days following ischemic surgery, when tissue oxygen tension had been restored, did not result in significant engraftment. This demonstrates that hypoxia-induced SDF-1 expression via HIF-1 is critically important in the selective homing and migration of CXCR⁴⁺ progenitor cells to ischemic tissues. This is the first demonstration that induction of SDF-1 expression via HIF-1 is able to directly guide regenerative progenitor cells to areas of injury.

### Example 22

### In Situ Examination of Bone Marrow Hypoxia

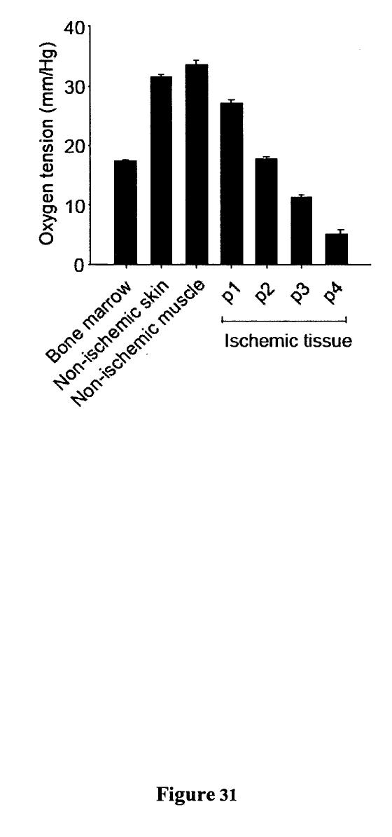

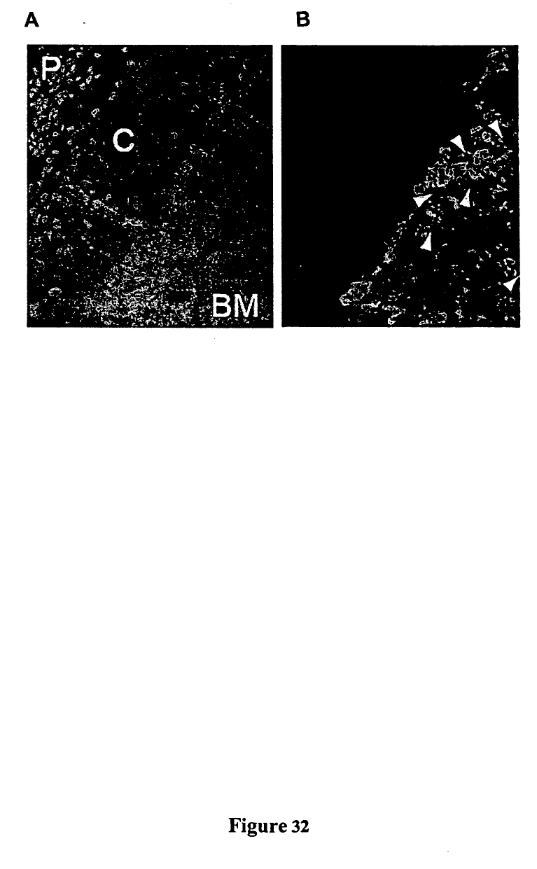

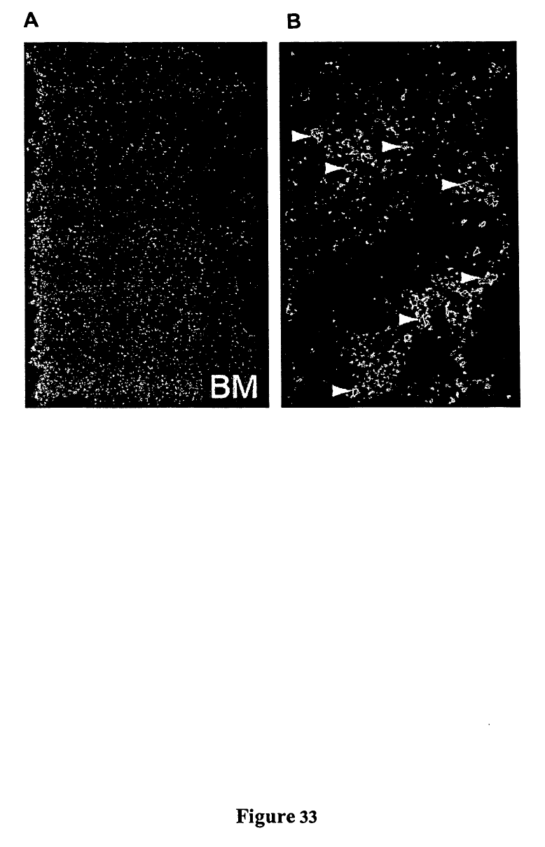

The results of Examples 15-21 raise the question of whether ischemia is required for progenitor cell localization, maintenance, and regeneration. If so, it would be expected that previously described stem and progenitor cell niches should be locally hypoxic. This has been suggested in a prior report indicating that bone marrow aspirates were hypoxic (Harrison et al., “Oxygen Saturation in the Bone Marrow of Healthy Volunteers,” Blood 99:394 (2002), which is hereby incorporated by reference in its entirety). Direct examination of the bone marrow and uninjured tissues of mice revealed that the oxygen tension in the bone marrow compartment in situ was consistently lower than other tissues, and in fact, strikingly similar to ischemic tissue in our model (see FIG. 31). As shown in FIGS. 32A-B, on a microscopic level, the bone marrow compartment contained discreet regions of hypoxia defined by pimonidazole localization that were associated with abundant SDF-1 immunostaining. Furthermore, systemically administered EPCs specifically homed to (day 2) and engrafted in (up to day 21) these regions, as shown in FIGS. 33A-B, regardless of the presence of a peripheral ischemic stimulus. Thus, heterogeneous regions of hypoxia in the bone marrow microenvironment may explain the constitutive and regional expression of SDF-1 and subsequent CXCR4-dependent stem and progenitor cell tropism (Peled et al., “The Chemokine SDF-1 Stimulates Integrin-mediated Arrest of CD34⁺ Cells on Vascular Endothelium Under Shear Flow,” J. Clin. Invest. 104:1199-1211 (1999), which is hereby incorporated by reference in its entirety).

In conjunction with the recent demonstration of CXCR4 regulation by HIF-1 (Staller et al., “Chemokine Receptor CXCR4 Downregulated by von Hippel-Lindau Tumour Suppressor pVHL,” Nature 425:307-311 (2003), which is hereby incorporated by reference in its entirety), the data from the experiments described in Examples 15-22 suggest that tissue hypoxia may be a fundamental mechanism governing stem and progenitor cell recruitment and retention. In this manner, transiently hypoxic microenvironments (such as injured tissue) may represent a conditional stem and progenitor cell niche where HIF-1 stabilization and activation of both the trafficking stimulus (SDF-1) and receptor (CXCR4) facilitates progenitor cell recruitment and retention within ischemic tissue requiring repair. This is supported by the dose-dependent relationship between absolute tissue oxygen tensions, SDF-1 expression and progenitor cell recruitment. Furthermore, these cells fail to localize in tissue after restoration of normal oxygen tension at which point SDF-1 levels have returned to the low steady state levels observed in uninjured tissues. This implies that progenitor cell-mediated tissue regeneration may require a locally hypoxic milieu for success. Thus, manipulation of HIF-1 activity may be a useful means to augment the body's innate reparative capacity (Elson et al., “Induction of Hypervascularity without Leakage or Inflammation in Transgenic Mice Overexpressing Hypoxia-inducible Factor-1α,” Genes Dev. 15:2520-2532 (2001), which is hereby incorporated by reference in its entirety). Furthermore, a reduction in HIF-1 activity, as occurs in aging (Rivard et al., “Age-dependent Defect in Vascular Endothelial Growth Factor Expression is Associated with Reduced Hypoxia-inducible Factor 1 Activity,” J. Biol. Chem. 275:29643-29647 (2000), which is hereby incorporated by reference in its entirety), may alter stem and progenitor cell trafficking and underlie the observed decline in regenerative capacity.

Here we demonstrate that SDF-1 gene expression is regulated by the transcription factor hypoxia-inducible factor-1 (HIF-1) in endothelial cells. This results in selective in vivo expression of SDF-1 in ischemic tissue in direct proportion to reduced oxygen tensions. Functionally, HIF-1 induced SDF-1 expression increases the adhesion, migration, and homing of circulating CXCR4⁺ progenitor cells to ischemic tissue. Blockade of SDF-1 in ischemic tissue or CXCR4 on circulating cells prevents progenitor cell recruitment to sites of injury. Furthermore, discrete regions of hypoxia in the bone marrow compartment exhibit increased SDF-1 expression and progenitor cell tropism. These data demonstrate that recruitment of CXCR4⁺ progenitor cells to regenerating tissues is mediated by hypoxic gradients via HIF-1 induction of SDF-1 expression.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. t,0460

What is claimed:

1. A method of treating damaged tissue, said method comprising:
increasing activity of hypoxia-inducible factor-1 and/or activity of stromal cell-derived factor-1 in damaged tissue to a level above that caused by damage alone to the tissue, under conditions effective to treat the damaged tissue.

2. The method according to claim 1, wherein the tissue is in vitro.

3. The method according to claim 1, wherein the tissue is in vivo.

4. The method according to claim 4, wherein the tissue is in a human patient.

5. The method according to claim 1, wherein the damaged tissue is selected from the group comprising ischemic tissue, tissue located at a wound site, and combinations thereof.

6. The method according to claim 1, wherein activity of hypoxia-inducible factor-1 is increased.

7. The method according to claim 1, wherein activity of stromal cell-derived factor-1 is increased.

8. The method according to claim 7, wherein activity of stromal cell-derived factor-1 is increased by increasing expression of stromal cell-derived factor-1.

9. The method according to claim 8, wherein expression of stromal cell-derived factor-1 is increased by increasing the activity hypoxia-inducible factor-1.

10. A method of modulating recruitment of stem cells and/or progenitor cells to a selected tissue site, said method comprising:
controlling activity of hypoxia-inducible factor-1 and/or activity of stromal cell-derived factor-1 at the selected tissue site to a level different than activity otherwise present at the selected tissue site, under conditions effective to modulate recruitment of stem cells and/or progenitor cells to the selected tissue site.

11. The method according to claim 10, wherein the tissue is in vitro.

12. The method according to claim 10, wherein the tissue is in vivo.

13. The method according to claim 12, wherein the tissue is in a human patient.

14. The method according to claim 10, wherein activity of hypoxia-inducible factor-1 is increased, whereby recruitment of stem cells and/or progenitor cells to the selected tissue site is increased.

15. The method according to claim 10, wherein activity of hypoxia-inducible factor-1 is decreased, whereby recruitment of stem cells and/or progenitor cells to the selected tissue site is diminished.

16. The method according to claim 10, wherein activity of stromal cell-derived factor-1 is increased, whereby recruitment of stem cells and/or progenitor cells to the selected tissue site is increased.

17. The method according to claim 16, wherein activity of stromal cell-derived factor-1 is increased by increasing expression of stromal cell-derived factor-1.

18. The method according to claim 17, wherein expression of stromal cell-derived factor-1 is increased by increasing activity of hypoxia-inducible factor-1.

19. The method according to claim 10, wherein activity of stromal cell-derived factor-1 is decreased, whereby recruitment of stem cells and/or progenitor cells to the selected tissue site is diminished.

20. The method according to claim 19, wherein activity of stromal cell-derived factor-1 level is decreased by decreasing expression of stromal cell-derived factor-1.

21. The method according to claim 20, wherein expression of stromal cell-derived factor-1 is decreased by decreasing activity of hypoxia-inducible factor-1.

22. The method according to claim 10, wherein the selected tissue site is selected from the group comprising a site where there is ischemia, a site where there is damaged tissue, a wound site, a tumor site, a site where there is tissue susceptible to damage, or combinations thereof.

23. The method according to claim 10, wherein CXCR4⁺ cells are recruited.

24. A method of treating cancer in a subject, said method comprising:
limiting activity of hypoxia-inducible factor-1 and/or activity of stromal cell-derived factor-1 in cancerous tumor tissue in the subject under conditions effective to treat the subject's cancer.

25. The method according to claim 24, wherein the subject is a human.

26. The method according to claim 24, wherein the cancer is selected from the group consisting of breast cancer, colon cancer, central nervous system cancer, leukemia, melanoma, lung cancer, ovarian cancer, prostate cancer, and renal cancer.

27. The method according to claim 24, wherein activity of hypoxia-inducible factor-1 is limited.

28. The method according to claim 24, wherein activity of stromal cell-derived factor-1 is limited.

29. The method according to claim 28, wherein activity of stromal cell-derived factor-1 is limited by limiting expression of stromal cell-derived factor-1.

30. The method according to claim 29, wherein expression of stromal cell-derived factor-1 is limited by limiting activity of hypoxia-inducible factor-1.
